(19) United States
(12) Patent Application Publication
(10) Pub. No.: US 2013/0267533 A1
(43) Pub. Date: Oct. 10, 2013

(54) 5HT1A AGONISTS FOR TREATMENT OF HIGH CHOLESTEROL

(71) Applicant: FABRE-KRAMER PHARMACEUTICALS, INC., Houston, TX (US)

(72) Inventors: Louis F. FABRE, Houston, TX (US); Stephen J. KRAMER, Houston, TX (US); Lou SMITH, Houston, TX (US)

(73) Assignee: FABRE-KRAMER PHARMACEUTICALS, INC., Houston, TX (US)

(21) Appl. No.: 13/841,161

(22) Filed: Mar. 15, 2013

Related U.S. Application Data
(60) Provisional application No. 61/635,545, filed on Apr. 19, 2012.
(60) Provisional application No. 61/620,218, filed on Apr. 4, 2012.

(51) Int. Cl. A61K 31/506

(52) U.S. Cl. CPC A61K 31/506
USPC 514/252.18; 514/252.14

(57) ABSTRACT

A method of treating and/or lowering elevated cholesterol by administering a therapeutically effective amount of at least one 5HT_{1A} receptor agonist to a subject in need thereof as either a short term or long term therapy. Also, a method of maintaining clinically acceptable cholesterol levels, or cholesterol levels below an elevated amount, by administering a therapeutically effective amount of at least one 5HT_{1A} receptor agonist to a subject in need thereof as either a short term or long term therapy.

### CROSS REFERENCE TO RELATED APPLICATIONS

This application claims priority to the following US Provisional Applications: U.S. 61/620,218 filed Apr. 4, 2012, and U.S. 61/635,545 filed Apr. 19, 2012, the texts of which are incorporated by reference.

### FIELD OF THE INVENTION

The present invention relates to a method of treating and/or lowering cholesterol levels in a patient in need thereof by administering a pharmaceutically effective amount of at least one 5HT_{1A} receptor agonist.

### BACKGROUND OF THE INVENTION

Major Depression and disorders associated therewith have been linked to early death due to cardiovascular mechanisms secondary to increased lipids and/or the metabolic syndrome. Psychiatric drugs have also been implicated in deleterious effects of weight, lipids and glucose. The 5HT_{2-D2} antagonists are especially damaging in this regard, with a significant proportion of subjects gaining weight, with complications of high lipid levels and diabetes. Although not wishing to be bound by any theory, it has been theorized that 5HT₂ receptor antagonism is responsible for these events. Furthermore, the literature suggests that cholesterol levels were low in patients with major depression (1, 2, 3, 4 and 5).

Psychiatric drugs also often increase cholesterol levels (6). The typical treatment for Major Depression is the Selective Serotonin Reuptake Inhibitors (SSRIs). Several studies have shown and the data in this application confirms that fluoxetine and sertraline elevate cholesterol levels (7, 8, 9).

Currently, statins (HMG-CoA reductase inhibitors) are a class of drugs used to lower cholesterol levels by inhibiting the enzyme HMG-CoA reductase, which plays a central role in the production of cholesterol in the liver (10). The best selling statin is atorvastatin, marked as Lipitor® and Torvast®. Rosuvastatin is also marketed as Crestor®. By 2003, atorvastatin became the best selling pharmaceutical in history, with Pfizer reporting sales of US $12.4 billion in 2008. On average, statins can lower LDL cholesterol by 1.8 mmol/L (70 mg/L), which translates into a 60% decrease in the number of cardiac events (heart attack, sudden cardiac death) and a 17% reduced risk of stroke after long-term treatment (11). The large decrease in cholesterol levels was accomplished in patients selected to have high initial cholesterol levels. However, statins have serious side effects which the 5HT_{1A} agonists do not have including muscle pain and wasting, liver damage, neurological side effects, allergic reactions, constipation, and birth defects. One serious side effect of statin drugs is unexpected muscle pain with fever. Statins can cause a condition that results in the breakdown of skeletal muscle tissue, rhabdomyolysis, and a more ominous threat of specific primary renal toxicity and death. Doctors must exercise special caution in the use of this drug with hypothyroidism, renal insufficiency, Asian sub-population groups and cyclosporine and gemfibrozil takers. In addition, liver damage is an uncommon but very serious side effect of statins. Anyone taking statins should undergo blood tests on a regular basis to ensure that the liver is not affected. Neurological side effects include but are not limited to headaches, dizziness, depression, numbness and tingling in the extremities, chronic trouble sleeping, memory loss and increased anxiety. Risk for the above neurological side effects is increased upon combination with other medications. Allergic reactions can include hives, general itching, rashes and swelling. Patients taking statins can also experience constipation, joint pain, nausea, and pregnant women should not take statins as the medication can cause birth defects.

There exists a need for pharmaceutical compounds that are effective at treating and/or lowering high cholesterol levels without imparting serious side effects as noted above.

The question of the impact of cholesterol on drug efficacy has also been investigated. With currently available antidepressant drugs, predominately SSRIs and SNRIs, only approximately one third of patients with major depression experience an adequate response (12). The reason for this is not known. There have been several attempts to try to understand which population will respond to antidepressants and which populations will not.

One study reported that fluoxetine does not work well in depressed subjects with high cholesterol levels (over 200 mg/dL)(13). The data suggests that above 200 mg/dL, the response rate (50% decrease from baseline HAMD-17) is 46%, and below 200 mg/dL, the response rate is 54%. The difference in response rates is statistically significant (p=0.05). Additionally, it was also reported that elevated total cholesterol was a significant predictor of treatment non-response and lack of remission (14, 15). It is postulated that there is a link between cholesterol homeostasis and serotonergic function (16-20).

However, the data from this study suggests several deficiencies. Firstly, the data is derived from an open label study. There is no placebo or other control group. Secondly, the higher cholesterol subjects differ in age from the lower cholesterol subjects: the average age of the elevated cholesterol group (n=166, mean 238.6±33.4 mg/dL) was 42.7; the average age of the lower cholesterol group (n=156, mean 170.4±22.2 mg/dL) was 36.8. The difference in age is significant at p<0.0001.

The question of whether high cholesterol levels effect the efficacy of 5HT_{1A} agonists has not been investigated.

### REFERENCE DOCUMENTS

(1) Wardle, J., Cholesterol and psychological well-being. J Psychosom Res 1995; 39:549-562.
(2) Borgherini G, Dorz S, Conforti D, et al. Serum cholesterol and psychological disease in hospital depressed patients. Acts Psychiatr Scand. 2002; 105:149-152.
(3) Olusi, S O, Fido A A. Serum lipid concentrations in patients with major depression. Biol Psychiatry 1996; 40:1128-1131.
(4) Oxenkrug, G F, Branconnier R J, Harto-Truax, N et. Al., Is serum cholesterol a biological marker for major depressive disorder.
(5) Tedders, S H, Fokung K D, MeKenzie L E at al, Low Cholesterol is associated with depression among US household populations. J Affective Disorders 2011; 135: 115-121.
(6) Thase, M E Corya S A, Osuntokun O, et al., A Randomized Double-Bind Comparison of Olansapine/Fluoxetine Combination, Olansapine, and Fluoxetine in Treatment Resistant Major Depressive Disorder. J Clin Psychiatry 2007; 68:224-236.
(7) Raeder M B, Bjelland I, Emil Vollset S, Steen V M. Obesity, dyslipidemia, and diabetes with selective serotonin reuptake inhibitors: the Hordaland Health Study. J Clin Psychiatry. 2006 December; 67:1974-82.
(8) Wei, F A, Crain L, Whitebird R R. Effects of Paroxetine and Seratraline on Low-Density Lipoprotein Cholesterol. CNS Drugs 2009; 23:857-865.
(9) Papakostas, G I, Iosifescu D V, Petersen, T et al. Serum Cholesterol in the Continuation Phase of Pharmacotherapy With Fluoxetine in Remitted Major Depressive Disorder. J Clin Psychopharm 2004; 24:467-469.
(10) Taylor, F., Ward, K., Moore, T H., et al. Statins for the primary prevention of cardiovascular disease. Cochrane Database Syst Rev. 2011; 1:CD004816.
(11) Kim E J, YU B-H., Increased Cholesterol levels After Paroxetine Treatment in Patients With Panic Disorder. Clinical Psychopharmacol 2005; 25:597-599.
(12) Trivedi M H, Daly E J. Treatment strategies to improve and sustain remission in major depressive disorder. Dialogues Clin Neurosci. 2008; 10:377-84.
(13) Sonawalla S B, Papakostas G I, Petersen T J, Yeung A S, Smith M M, Sickinger A H, Gordon J, Israel J A, Tedlow J R, Lamon-Fava S, Fava M. Elevated cholesterol levels associated with nonresponse to fluoxetine treatment in major depressive disorder. Psychosomatics, 2002; 43:310-6.
(14) Papakostas G I, Petersen T, Sonawalla S B, Merens W, Iosifescu D V, Alpert J E, Fava M, Nierenberg A A, Serum cholesterol in treatment-resistant depression. Neuropsychobiology. 2003; 47:146-51.
(15) Papakostas G I, Iosifescu D V, Paterson T, Hamill S K, Alpert J E, Nierenberg A A, Rosenbaum J F, Fava M. Serum cholesterol in the continuation phase of pharmacotherapy with fluoxetine in remitted major depressive disorder. J Clin Psycopharmacol. 2004; 24:467-9.
(16) Scanlon S M, Williams D C, Schloss P. Membrane cholesterol modulates serotonin transporter activity. Biochemistry. 2001; 40:10507-13.
(17) Buydens-Branchey L, Branchey M, Hundson J., Fergeson P. Low HDL cholesterol, aggression and altered central serotonergic activity. Psychiatry Res. 2000; 93:93-102.
(18) Iosifescu D V, Clementi-Craven N, Fragaus R, Papakostas G I, Petersen T, Alpert J E, Nierenberg A A, Fava M., Cardiovascular risk factors may moderate pharmacological treatment effects in major depressive disorder. Psychocom Med. 2005; 67:703-6.
(19) Pucadyil T J, Chattopadhaya A, Cholesterol modulates the antagonist-binding function of hippocampal serotonin1A receptors. Biochem Biophys Aceta 2005; 1714:35-42.
(20) Mossner R, Mikova O, Koutsilieri E, Saoud M, Ehlis A C, Muller N, Fallgatter A J, Riederer P. Consensus paper of the WFSBP Task Force in Biological Markers: biological markers in depression. World J Biol Psychiatry. 2007; 8:141-74.

### SUMMARY OF INVENTION

The above object highlights certain aspects of the invention. Exemplary embodiments of the present invention include, but are not limited to;

(1) A method of treating an elevated cholesterol level comprising administering a therapeutically effective amount of at least one 5HT_{1A} receptor agonist to a subject in need thereof.

(2) The method of paragraph (1) above, wherein the 5HT_{1A} receptor agonist is at least one selected from the group consisting of gepirone, ipsapirone, tandospirone, adatanserin, flesinoxan or buspirone.

(3) The method of paragraph (1) above, wherein the subject in need thereof also suffers from and/or is diagnosed with at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's.

(4) The method of paragraph (1) above, wherein the 5HT_{1A} receptor agonist is gepirone.

(5) The method of paragraph (1) above, wherein the 5HT_{1A} receptor agonist is adatanserin.

(6) The method of paragraph (1) above, wherein the elevated cholesterol level is a cholesterol level that exceeds 200 mg/dL.

(7) The method of paragraph (1) above, wherein said subject is human.

(8) The method of paragraph (1) above, wherein said therapeutically effective amount ranges from 20-80 mg per day per day.

(9) A method of maintaining a cholesterol level below 200 mg/dL in a subject in need thereof, said method comprising administering a therapeutic effective amount of at least one 5HT_{1A} receptor agonist to said subject.

(10) The method of paragraph (9) above, wherein the 5HT_{1A} receptor agonist is at least one selected from the group consisting of gepirone, ipsapirone, tandospirone, adatanserin, flesinoxan and buspirone.

(11) The method of paragraph (9) above, wherein the subject in need thereof suffers from and/or is diagnosed with at least one of the following disorders selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's.

(12) The method of paragraph (9) above, wherein the 5HT_{1A} receptor agonist is gepirone.

(13) The method of paragraph (9) above, wherein the 5HT_{1A} receptor agonist is adatanserin.

(14) The method of paragraph (9) above, wherein said subject is human.

(15) The method of paragraph (9) above, wherein said therapeutically effective amount ranges from 20-80 mg per day.

(16) A method of treating at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's, said method comprising administering to a subject in need thereof an effective amount of at least one 5HT_{1A} agonist, wherein the subject has an elevated cholesterol level.

(17) The method of paragraph (16) above, wherein the 5HT_{1A} receptor agonist is at least one selected from the group consisting of gepirone, ipsapirone, tandospirone, adatanserin, flesinoxan and buspirone.

(18) The method of paragraph (16) above, wherein an elevated cholesterol level is a cholesterol level that exceeds 200 mg/dL.

(19) A method of treating at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's, said method comprising:

identifying a subject suffering from at least one of the above disorders that has an elevated cholesterol level,

administering to a subject in need thereof an effective amount of at least one 5HT_{1A} agonist.

(20) The method of paragraph (19) above, wherein the 5HT_{1A} receptor agonist is at least one selected from the group consisting of gepirone, ipsapirone, tandospirone, adatanserin, flesinoxan and buspirone.

(21) The method of paragraph (19) above, wherein an elevated cholesterol level is a cholesterol level that exceeds 200 mg/dL.

Additional objects, aspects and embodiments of the invention are found in the following detailed description of the invention.

### BRIEF DESCRIPTION OF THE DRAWINGS

A more complete appreciation of the invention and many of the attendant advantages thereof will be readily obtained as the same becomes better understood by reference to the following figures in conjunction with the detailed description below.

FIG. 1: The 5-HT (serotonin) neuron in the hyperactive state: namely, high levels of 5-HT, overactive 5-HT neuronal firing and transmission, and down-regulated somatodendritic presynaptic 5-HT_{1A} autoreceptors.

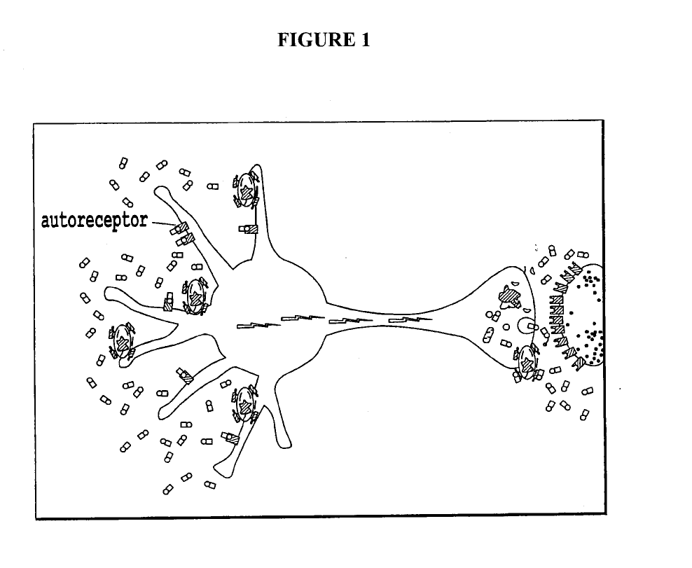

FIG. 2: The drugs (gepirone, ipsapirone, tandospirone, flesinoxan, and adatanserin) are presynaptic agonists and post-synaptic partial agonists. A presynaptic agonist shuts off the neuron (less serotonin post-synaptically). Post-synaptic partial agonism results in less robust stimulation of the post-synaptic neuron than serotonin itself (resulting in less firing). The net result is less post-synaptic neuron firing.

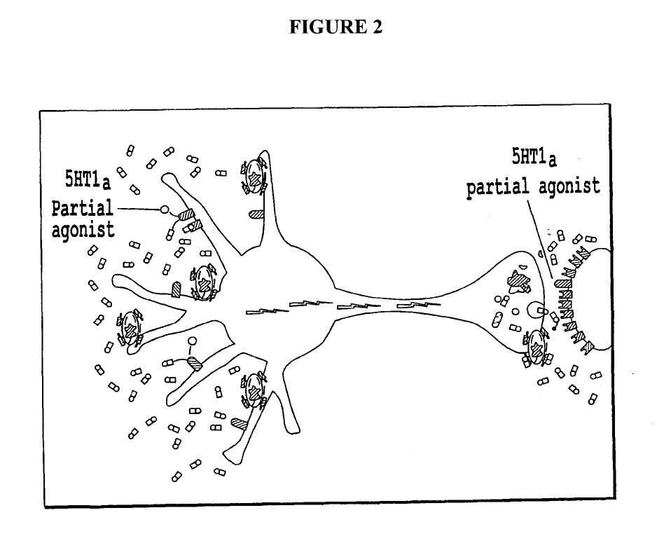

FIG. 3: Meta Analysis of cholesterol levels in major depression comparing SSRIs to placebo.

FIG. 4: Meta Analysis of cholesterol levels in major depression comparing SSRIs to placebo.

FIG. 5: Meta Analysis of cholesterol levels in major depression comparing SSRIs to Gepirone-ER.

FIG. 6: Meta Analysis of effect of Gepirone on total cholesterol levels.

FIG. 7: Meta Analysis of effect of Gepirone on total cholesterol levels in 40-59 year old patients.

### DETAILED DESCRIPTION OF THE INVENTION

Unless specifically defined, all technical and scientific terms used herein have the same meaning as commonly understood by a skilled artisan in organic chemistry, biochemistry, psychology, psychiatry, medicine, neurochemistry, and neurology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention relates to (1) the surprising discovery that cholesterol levels can be lowered by administering at least one 5HT_{1A} receptor agonist to a subject in need thereof; and (2) the surprising discovery that elevated cholesterol levels increase the efficacy of 5HT_{1A} receptor agonists for treatment of a plurality of disorders including but not limited to attention deficit disorder, depression, sexual dysfunction, psychoses, aggressive behavior, alcohol dependence, obesity, anxiety, drug abuse/addiction, sleep disorders, and behavioral/cognitive symptoms of Alzheimer's.

(1) Method of Treating and/or Lowering Cholesterol Levels

The present invention relates to a method of treating and/or lowering cholesterol levels in a patient in need thereof by administering a pharmaceutically effective amount of at least one 5HT_{1A} receptor agonist.

The literature suggests that cholesterol levels were low in patients with major depression. However, depressed patients typically die 10-20 years earlier than non depressed patients largely due to cardiovascular disease. Therefore, the inventors of this invention questioned this literature and investigated total cholesterol levels in 10 studies of gepirone at baseline before any treatment (See Examples 1-2). It was surprisingly discovered that in this population of depressed subjects cholesterol levels were increased.

The present inventors further studied the effect of 5-HT_{1A} agonists on cholesterol levels and it was surprisingly found that the 5-HT_{1A} agonists decreased cholesterol levels in a clinically significant amount. This was not expected in view of the above studies of SSRIs. Specifically, as noted above in patients with major depression, treatment with SSRI's results in increased cholesterol levels, whereas the present invention is based, in part, on the discovery that treatment with 5HT_{1A} agonists results in statistically significant decreases in cholesterol levels (See Examples 3-4).

5HT_{1A} agonists are essentially free of the adverse events imparted by statins other than limited mild dizziness and nausea. 5HT_{1A} agonists are not known to inhibit HMG-CoA reductase, nor do they have chemical structures that are similar to statins. While not wishing to be bound by any theory, it is theorized that 5HT_{1A} agonists reduce cholesterol by another mechanism than statins.

The term “elevated cholesterol level” means a cholesterol level above 240 mg/dL, preferably above 200 mg/dL, more preferably above 150 mg/dL, more preferably above 120 mg/dL, more preferably above 100 mg/dL.

The lowering effect of 5HT_{1A} agonists on lipid levels will improve quality and duration of life in depressed patients.

(2) Method of Treating Disorders with 5HT_{1A} Agonists in Subjects Having Elevated Cholesterol Levels

Another embodiment of the present invention relates to the discovery of a direct connection between serotonergic receptor function and cholesterol levels. The present inventors examined whether high cholesterol levels negatively impacts the effects of 5HT_{1A} agonists and surprisingly found that the effects of 5HT_{1A} agonists are increased in subjects with high cholesterol (Example 5). This improved effect was not expected in view of the above Fava studies, and provides a method of treatment wherein subjects with higher cholesterol levels may be pre-screened and in turn improve the efficacy of the administered 5HT_{1A} agonists.

### 5HT_{1A} Receptor Agonists

The term “5-HT_{1A} receptor agonist” means partial agonist or full agonist with respect to either presynaptic receptors or postsynaptic receptors, or both, unless otherwise specified.

In psychopharmacology, the serotonin (5-hydroxytryptamine [5-HT]) type-1A (5-HT_{1A}) receptor has acquired considerable attention as a therapeutic target. Agonism of the 5-HT_{1A} receptor has been correlated with treatment of anxiety, depression, obesity, drug abuse/addiction, alcohol abuse, sleep disorders and behavioral and cognitive symptoms of Alzheimer's disease (U.S. Pat. No. 4,423,049, U.S. Pat. No. 4,771,053, U.S. Pat. No. 5,106,849, and Bison, Journal of Clinical Psychopharma, 1990; 10: S2-S5).

The 5HT_{1A} receptor agonists which may be used according to the claimed invention can be administered alone or with one or more other 5HT_{1A} receptor agonists. Examples of the 5HT_{1A} receptor agonists include gepirone (also known as 4,4-dimethyl-1-[4-[4-(2-pyrimidinyl)1-piperazinyl]-butyl]-2,6-piperadinedione hydrochloride), ipsapirone (2-(4-(4-(2-pyrimidinyl)-1-piperazinyl)-butyl) 1,2-benzoisothiazol-3(2H)-one 1,1-dioxide hydrochloride), tandospirone (N-[4-[4-(2-pyrimidinyl)-1-piperazinyl]butyl]-2,3-norbornanedicarboximide), adatanserin (N-[2-[4-(2-pyrimidinyl)-1-piperazinyl]ethyl]tricyclo[3.3.1.1^{3,7}]decane-1-carboxamide), Flesinoxan ((+)-N-[2-[4-(2,3-dihydro-2-hydroxymethyl-1,4-benzodioxin-5-yl)-lpiperaxinyl]ethyl]-4-fluorobenzamide hydrochloride), or buspirone (8-[4-4-pyrimidin-2-ylpiperazin-1-yl)butyl]-8-azaspiro[4.5]decane-7,9-dione). The term “5-HT_{1A} receptor agonist as used herein also embraces the bioactive metabolites of the aforementioned compounds. Particularly preferred are the hydroxyl metabolites. Particularly preferred are the 3-OH gepirone and 3-OH adatanserin, 5-OH gepirone, and 3,5-dihydroxy gepirone, with 3-OH gepirone being the most preferred metabolite.

The 5HT_{1A} receptor agonist may be administered in an immediate release formulation, or an extended release formulation. Examples of the immediate release formulation are disclosed in the following references which are incorporated by reference: U.S. Pat. No. 4,423,049, U.S. Pat. Nos. 4,782,060, and 5,106,849. Examples of the extended release formulation are disclosed in the following references which are incorporated by reference: U.S. Pat. No. 5,478,572, US 2006/0099267, and US 2005/0095286.

The pharmaceutical compounds suitable for administration in the present invention may be hydrochloride salts, but the free bases and other pharmaceutically acceptable salts are also suitable. The term “pharmaceutically acceptable salt” is well known in the art, as described in S. M. Berge et al. (J Pharmaceutical Sciences, 66: 1-19, 1977). Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably nontoxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, loweralkyl sulfonate and aryl sulfonate.

A therapeutically effective amount of the pharmaceutical compounds suitable for administration in the present invention may be administered alone or in combination with one or more pharmaceutically acceptable carriers. As used herein, the term “pharmaceutically acceptable carrier” means a non-toxic, inert solid, semi-solid or liquid filer, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions suitable for administration in the invention can be administered to patients in need thereof orally, rectally, nasally, parenterally (e.g., intramuscular, intraperitoneal, intravenous or subcutaneous injection, or implant), intracisternally, intravaginally, intraperitoneally, sublingually, topically (e.g., as a powder, ointment, or drop), bucally, as an oral spray, or a nasal spray. The pharmaceutical compositions can be formulated in dosage forms appropriate for each route of administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art. The inert diluents may include, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The liquid dosage form for oral administration may also contain adjuvants, which include wetting agents, emulsifying and suspending agents, sweetening, flavoring, and perfuming agents. Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example arachis oil.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This maybe accomplished by the use of a liquid suspension of crystalline or amorphous material with poor water solubility. The rate of absorption of the drug then depends upon its rate of dissolution, which, in turn, may depend upon crystal size and crystalline form. Alternatively, dissolving or suspending the drug in an oil vehicle accomplishes delayed absorption of a parenterally administered drug form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, prills, powders, and granules. In such solid dosage forms, the active compound is mixed with at least one inert, pharmaceutically acceptable excipient or carrier. In addition, the solid dosage form may contain one or more fillers, extenders, binders, humectants, disintegrating agents, retarding agents, absorption accelerators, wetting agents, absorbents, or lubricants. Examples of suitable fillers or extenders include, starches, lactose, sucrose, glucose, mannitol, and silicic acid, sodium citrate and dicalcium phosphate. Examples of suitable binders include, microcrystalline cellulose, carboxymethylcellulose, alginates, gelatin, polyvinylpyrrolidinone, sucrose, and acacia. Glycerol is an example of a suitable humectant. Examples of suitable disintegrating agents include, agar-agar, calcium carbonate, potato or tapioca starch, maize starch, alginic acid, certain silicates, and sodium carbonate. Paraffin is an example of a suitable solution-retarding agent. As absorption accelerators, any quaternary ammonium compound may be used. Examples of suitable wetting agents include, cetyl alcohol and glycerol monostearate. Examples of suitable absorbents include, kaolin and bentonite clay. Examples of suitable lubricants include, talc, calcium stearate, magnesium stearate, solid polyethylene glycols, sodium lauryl sulfate. In the case of capsules, tablets and pills, the dosage form may also comprise buffering agents.

The tablets may, if desired, be coated using known methods and excipients that may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hardfilled gelatin capsules using such excipients as lactose or milk sugar as well as high molecular weight polyethylene glycols.

If desired, the compounds of the present invention can be incorporated into slow release or targeted delivery systems such as polymer matrices, liposomes and microspheres. They may be sterilized, for example, by filtration through a bacteria-retaining filter, or by incorporating sterilizing agents in the form of sterile solid compositions that can dissolve in sterile water, or some other sterile injectable medium immediately before use.

The active compound may be formulated into granules with or without additional excipients. The granules may be ingested directly by the patient or they may be added to a suitable liquid carrier (for example, water) before ingestion. The granules may contain disintegrates, e.g. an effervescent couple formed from an acid and a carbonate or bicarbonate salt to facilitate dispersion in the liquid medium.

Dosage forms for topical or transdermal administration of a compound of this invention include ointments, pastes, creams, lotions, gels, powders, solutions, sprays, inhalants or patches. Transdermal patches have the added advantage of providing controlled delivery of a compound to the body. The rate can be controlled by either providing a rate controlling membrane or by dispersing the compound in a polymer matrix or gel. The active component is admixed under sterile conditions with a pharmaceutically acceptable carrier and any needed preservatives or buffers as may be required. Dissolving or dispensing the compound in the proper medium can make such dosage forms. Absorption enhancers can also be used to increase the flux of the compound across the skin. Ophthalmic formulation, eardrops, eye ointments, powders and solutions are also contemplated as being within the scope of this invention.

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as animal and vegetable fats, oils, petrolatum, waxes, paraffins, starch, tragacanth, cellulose derivatives, polyethylene glycols, silicones, bentonites, silicic acid, talc and zinc oxide, or mixtures thereof, together with a potential transdermal accelerant such as dimethyl sulphoxide or propylene glycol. Alternatively the active compounds may be dispersed in a pharmaceutically acceptable paste, cream, gel or ointment base. The amount of active compound contained in a topical formulation should be such that a therapeutically effective amount of the compound is delivered during the period of time for which the topical formulation is intended to be on the skin.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. The therapeutically active compound may be formulated into a composition, which is dispersed as an aerosol into the patient's oral or nasal cavity. Such aerosols may be administered from a pump pack or from a pressurized pack containing a volatile propellant.

The therapeutically active compounds used in the method of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

The 5-HT_{1A} agonist may be employed in conjunction with an agent selected from the group consisting of stimulants, hypnotics, anxiolytics, antipsychotics, antianxiety agents, minor tranquilizers, benzodiazepines, barbituates, serotonin agonists, selective serotonin reuptake inhibitors, dopamine antagonists, 5-HT_{1A} agonists, 5-HT₂ antagonists, non-steroidal anti-inflammatory drugs, monoamine oxidase inhibitors, muscarinic agonists, norephinephrine uptake inhibitors, essential fatty acids, and neurokinin-1 receptor antagonist.

As used herein, the term “therapeutically effective amount” refers to that amount of a compound or preparation of the present invention that successfully reduces elevated cholesterol levels. This term also embraces the amount of a compound or preparation of the present invention that successfully reduces the severity of symptoms associated with attention deficit disorder, with or without hyperactivity, depression, sexual dysfunction, psychoses, aggressive behavior, alcohol dependence, obesity, anxiety, drug abuse/addiction, sleep disorders, and behavioral/cognitive symptoms of Alzheimer's.

It is contemplated that the therapeutically effective amount of a composition will depend on a number of factors, including by not limited to the age of the patient, immune status, race, and sex of the patient, and the severity of the condition/disease, and the past medical history of the patient, and always lies within the sound discretion of the administering physician. Generally, the total daily dose of the 5HT_{1A} receptor agonist of this invention administered to a patient in single or in divided doses can be in amounts, for example, of 15 to 90 mg/day, preferably 20-80 mg/day, or about 0.01 to 40 mg/kg body weight/day, preferably 0.05 to 20 mg/kg body weight/day, more preferably 0.1 to 2 mg/kg of body weight/day. See U.S. Pat. No. 4,771,053 and U.S. Pat. No. 5,478,572. Alternatively, the effective dose or delivery system should result in plasma concentrations in the range of about 1 ng/ml to about 20 ng/ml, preferably about 1 ng/ml to about 5 ng/ml.

The above disclosed range is intended to encompass the ranges disclosed therein including subranges from each individual point within said range to any other individual point within said range.

Single dose compositions may contain such amounts or submultiples thereof to make up the daily dose. Treatment regimens according to the present invention also include concurrently administering to a patient in need thereof mixtures, in single or divided doses, either simultaneously or at different times, of two or more of the compounds of the present invention in an effective amount. When the compounds of the present invention are administered concurrently as mixtures, the therapeutically effective amount to be administered lies within the sound discretion of the administering physician.

Alternatively, treatment regimens according to the present invention include sequentially administering to a patient in need thereof, in single or divided doses, two or more of the compounds of the present invention. An example of a sequential administration strategy includes administering a therapeutically effective amount of a first compound followed by, on the same day or a subsequent day, a single or divided dose of a therapeutically effective amount of one or more additional compounds. As used herein, the term “subsequent day” refers to any day ranging from the next day (>24 hours) to one week (<168 hours) after administration of the previous compound. The term “same day” refers to any time frame ranging from immediately after administration of the previous compound to <24 hours after administration of the previous compound.

As used herein, the terms “treat”, “treating”, and “treatment” also embrace the terms alleviation and amelioration of elevated cholesterol levels. Moreover, the terms “treat”, “treating”, and “treatment” also may embrace reduction of elevated cholesterol levels as well as symptoms associated therewith. i.e administration to a subject that does not have elevated cholesterol levels in order to maintain said levels at a low amount.

As used herein, the term “prevent,” and “preventing” embrace reducing the likelihood of developing elevated cholesterol by at least 5%, preferably at least 10%, more preferably at least 20%, more preferably at least 30%, more preferably at least 40%, more preferably at least 50%, more preferably at least 60%, more preferably at least 70%, more preferably at least 80%, most preferably at least 90%. Preventing can also mean that while the active agent is being administered the subject does not have an increase in cholesterol by more than 50 mg/dL, preferably not more than 40 mg/dL, more preferably not more than 30 mg/dL, more preferably not more than 20 mg/dL, more preferably not more than 10 mg/dL.

As used herein, the term “subject” embraces all mammals, in particular humans.

The following is a description of exemplary 5HT_{1A} agonists and extended release formulations thereof.

### Gepirone

Gepirone (also known as 4,4-dimethyl-1-[4-[-(2-pyrimidinyl)-1-piperazinyl]-butyl]-2,6-piperidinedione hydrochloride) can be obtained by the process described in Example 7 of Temple, U.S. Pat. No. 4,423,049 (which is incorporated herein in its entirety by reference) and has the following structure:

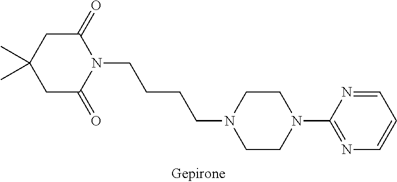 [SMILES: CC1(C)CC(=O)N(CCCCN2CCN(c3ncccn3)CC2)C(=O)C1]

Gepirone is an azapirone 5-HT_{1A} partial agonist. The drug has been shown effective in the treatment of anxiety, depression, and sexual dysfunction (See U.S. Pat. No. 6,534,507; U.S. Pat. No. 6,835,728; U.S. Pat. No. 7,538,116; the texts of which are incorporated herein by reference).

3-OH-Gepirone is the major metabolite of gepirone. 3-OH-Gepirone is also a partial agonist of 5-HT_{1A}. 3-OH-Gepirone has the same chemical structure as gepirone except for a hydroxyl group in the 3 position

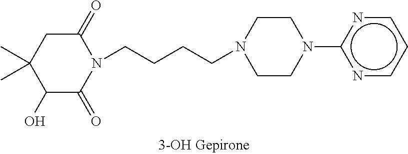

(See U.S. 2009/0281111 the text of which is incorporated herein by reference). References to gepirone in this specification encompass metabolites thereof, including 3-OH-gepirone metabolite.

The gepirone used in the method of the present invention can be either extended release or immediate release.

Extended release drug forms offer several advantages over immediate release systems. Patient compliance is better because the extended release dosage forms need to be taken only one in a 24 hour period. Thus, plasma concentration levels do not vary unacceptably—i.e. give high initial drug levels that are associated with the incidence of unwanted side effects, as well as having rapid drops in drug levels to below therapeutic levels—when the ER dosage forms are administered. In addition, in the case of gepirone, due to the slow time release 1-PP levels are maintained at a satisfactory level thus avoiding significant complications arising from this metabolite.

In one possible embodiment, the present invention comprises administering an oral extended release gepirone low-dosage composition comprising:
(1) from about 0.5 to 12 wt % gepirone, more preferably 0.6 to 10.7 wt % gepirone or bioactive metabolite thereof, as a free base or a pharmaceutically acceptable salt thereof;
(2) from about 70 to about 85 wt % of a pharmaceutically acceptable cellulosic polymer matrix; and
(3) suitable amounts of one or more pharmaceutically acceptable excipients, wherein the release rate of gepirone from the dosage from is such that about 18 to 24 hours are required to attain from about 90 to about 95% absorption of gepirone.

In another possible embodiment, the present invention comprises administering an oral extended release gepirone high-dosage composition comprising:
(1) from about 14 to about 25 wt % gepirone, or bioactive metabolite thereof, as a free base or a pharmaceutically acceptable salt thereof;
(2) from about 70 to about 85 wt % of a pharmaceutically acceptable cellulosic polymer matrix; and
(3) suitable amounts of one or more pharmaceutically acceptable excipients, wherein the release rate of gepirone from the dosage from is such that about 18 to 24 hours are required to attain from about 90 to about 95% absorption of gepirone.

In another possible embodiment, the present invention comprises administration of an extended release oral dosage form of gepirone, containing, in percentages expressed by weight:
(a) about 14.0 to about 24.4% (preferably, about 15.5 to about 18.7%) gepirone, or bioactive metabolite thereof, as a free base or a pharmaceutically acceptable salt thereof, for example a hydrochloride salt,
(b) about 70.5 to about 82.1% hydroxypropylmethylcellulose having a viscosity of from about 15,000 to about 100,000 cps.,
(c) 0 to about 1% (preferably 0 to about 0.3%) iron oxide,
(d) about 8.0 to about 16.7% microcrystalline cellulose,
(e) about 0.39 to about 0.47% (preferably about 0.42 to about 0.47%) colloidal silica, and
(f) about 0.29 to about 1.0% magnesium stearate.

Although the above embodiments are defined in terms of particular compounds for each component, it is to be understood that within the scope of the present invention these components may be replaced individually or in various combinations as described below. For each of the components below, it is to be understood that the recited weight percentage reflects the total concentration of each component. For example, if there is a mixture of compounds that fall within the scope of a defined component group the recited weight percentage reflects the total for that mixture of compounds.

Component (a)

In a particular embodiment, component (a) is present in the extended-release oral dosage form in a weight percentage of about 14.0 to about 24.4%, more preferably about 15.5. to about 18.7%. It is particularly preferred that component (a) be gepirone or a pharmaceutically acceptable salt thereof.

In vivo, gepirone is metabolized resulting in to major pharmaceutically active metabolites: 1-(2-pyrimidinyl)piperazine (1-PP) and 3′-OH gepirone (D. S. Robinson et al, Clinical Therapeutics, pp. 1618-1633 (2003)). Two additional metabolites that may also possess bioactivity are also formed in vivo: 5-OH gepirone and 3′,5-dihydroxy gepirone.

In humans, the release of 1-PP, a common azapirone metabolite, is believed to be responsible for adverse side effects, including dizziness, nausea, headache, and drowsiness. Moreover, 1-PP is a presynaptic α-2-adrenoceptor antagonist and it has been reported that it did not exhibit antidepressant-like characteristics in pre-clinical tests (D. S. Robinson et al, Clinical Therapeutics, pp. 1618-1633 (2003)). In contrast, the bioactive metabolites (3′-OH gepirone, in particular) have significant affinity for 5-HT receptors. 3′-OH gepirone has been demonstrated to modify 5-HT neurotransmission in a comparable manner to gepirone even though 3′-OH has been found to exhibit full agonism post-synaptic receptors in the hippocampus, whereas gepirone is a partial agonist (D. S. Robinson et al, Clinical Therapeutics, pp. 1618-1633 (2003)).

In view of the foregoing, in an embodiment of the present invention one or more of the bioactive gepirone metabolites 3′-OH gepirone, 5-OH gepirone, and 3′,5-dihydroxy gepirone, or a pharmaceutically acceptable salt thereof, may be used in place of gepirone.

Further, although the pharmaceutically acceptable salt form is preferred, gepirone or the bioactive gepirone metabolites may be in a hydrate form, an enantiomeric form or mixture, or crystal form.

The pharmaceutical compounds suitable for administration in the present invention may be hydrochloride salts, but the free bases and other pharmaceutically acceptable salts are also suitable. The term “pharmaceutically acceptable salt” is well known in the art and the artisan is directed to S. M. Berge, (J. Pharmaceutical Sciences, 66: 1-9 (1997), incorporated herein by reference) for a further description. Suitable pharmaceutically acceptable salts for administration in the present invention include acid addition salts. The acid addition salt may be formed by mixing a solution of the compounds with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carried an acidic group, for example a carboxylic acid group, the present invention also contemplates salts thereof, preferably non-toxic pharmaceutically acceptably salts thereof, such as sodium, potassium, and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethane-sulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the qua-ternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, lower alkyl sulfonate, and aryl sulfonate.

Component (b):

In a preferred embodiment, component (b) is present in the extended-release oral dosage form in a weight percentage of about 70.5 to about 82.1%. Preferably, component (b) is hydroxypropylmethylcellulose having a viscosity of from about 15,000 to about 100,000 cps, which is a release-controlling polymer & binder (a.k.a., sustaining agent).

For oral formulations and dosage forms, the use of a polymeric cellulose matrix, or sustaining agent, is preferred. Suitable matrixes include hydroxyalkylsubstituted alkylcelluloses having viscosities of about 15,000 cps to about 100,000 cps. Examples of acceptable hydroxymethyl propylcellulose (HPMC) samples include grades KI5M and KIOOM (i.e., 15,000 and 100,000 cps, respectively).

HPMC may be replaced (all or in part) or added to in the present invention, the replacement of some or the entire HPMC matrix may be with dicalcium phosphate or lactose, each of which generally increases dissolution rates.

In addition to the binder of component (b), the formulation of the present invention may also contain auxiliary binding agents, such as syrup, acacia, gelatin, sorbitol, tragacanth, or polyvinylpyrrolidone. Examples of suitable fillers or extenders also include starches (e.g., maize-starch), lactose, sorbitol, glycine, sucrose, glucose, mannitol, and silicic acid, sodium citrate and dicalcium phosphate. Examples of other suitable binders may include chitosan, alginates, gelatin, polyvinylpyrrolidinone, sucrose, acacia, and mixtures thereof.

In an embodiment of the present invention the ratio of gepirone to binder (gepirone:binder) ranges from 1:3.5 to 1:14.5. Preferably, the binder is hydroxymethylpropylcellulose (HPMC).

Component (c):

In a preferred embodiment, component (c) is present in the extended-release oral dosage form in a weight percentage of 0 to about 1%, preferably 0 to about 0.3%. Although component (c) may be any colorant, it is preferred that the colorant be an iron oxide. In a particularly preferred embodiment the iron oxide is red ferric oxide, yellow ferric oxide, or mixtures thereof.

Additional exemplary colorants that may be used in place of or in addition to the foregoing include, but are not limited to, FD&C and D&C lakes, titanium dioxide, iron oxides, natural pigments, or dyes approved for ingestion by the U.S. Federal Drug Administration, or combinations thereof.

Component (d):

In a preferred embodiment, component (d) is present in the extended-release oral dosage form in a weight percentage of about 8.0 to about 16.7%. Although component (d) may be any diluent and/or compression aid, it is preferred that the diluent/compression aid be microcrystal-line cellulose.

Additional diluents may include, but are not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

In yet another embodiment of the present invention, the ratio of gepirone to diluent (gepirone:diluent) ranges from 1:3 to 2.5:1. Preferably, the diluent is microcrystalline cellulose.

Component (e):

In a preferred embodiment, component (e) is present in the extended-release oral dosage form in a weight percentage of about 0.39 to about 0.47%, preferably about 0.42 to about 0.47%. Although component (e) may be any glidant, it is preferred that the glidant be colloidal silica (colloidal silicon dioxide).

Additional glidants include, but are not limited to, cornstarch, talc, or stearic acid, or combinations thereof.

Component (f):

In a preferred embodiment, component (f) is present in the extended-release oral dosage form in a weight percentage of about 0.29 to about 1.0%. Although component (f) may be any lubricant, it is preferred that the lubricant be magnesium stearate.

Additional lubricants may include, but are not limited to cellulose, talc, polyethylene glycol, silicas, sodium lauryl sulfate, calcium stearate, and mixtures thereof.

### Alternative Components:

It is contemplated in the present invention that the extended-release oral dosage form may also contain, in addition to components (a) through (f), various additional components. Based on the description above and Remington's Pharmaceuticals Sciences, 18th Edition (incorporated herein by reference), especially Part 8 therein, “Pharmaceutical Preparations and Their Manufacture, it would be well within the purview of the skilled artisan to add new compounds to the preferred embodiments described above.

In an embodiment of the present invention, the extended-release oral dosage form may include one or more additional pharmaceutically acceptable carriers. As used herein, the term “pharmaceutically acceptable carrier” means a non-toxic, inert solid, semi-solid or liquid filer, diluent, encapsulating material or formulation auxiliary. Some examples of materials which can serve as pharmaceutically acceptable carriers are already mentioned above, therefore, it is understood that in this aspect of the invention, pharmaceutically acceptable carriers may be any type of filer, diluent, encapsulating material or formulation auxiliary not previously mentioned.

In another embodiment, the extended-release oral dosage form may include may also include one or more releasing agents, coating agents, sweetening, flavoring and perfuming agents, preservatives and antioxidants.

The flavorant(s), which is used primarily for taste- and/or odor-masking, may be vanillin, sodium citrate, citric acid, mint, orange, lemon oil, or any other pharmaceutically approved flavorant or tastemasking agent, and combinations thereof.

Shape And Physical State Of The High-Dosage Extended-Release Form Of Gepirone

The present invention contemplates extended-release oral dosage forms that are suitable for orally administration. These forms include tablets, capsules, caplets, lozenges, powders, suspensions, syrups and the like are suitable forms. Preferentially, the tablet may be mentioned including those having a convex shape, a spherical (i.e., round) shape, or a capsule shape.

In a preferred embodiment, the extended-release oral dosage form of gepirone is a tablet. Preferably, the tablet has oval shape, which enhances their surface area and improves the release of gepirone therefrom. More preferably, the tablet has an ovoid-rectangular shape, which may have either flat or biconvex faces.

When the extended-release oral dosage form of gepirone is a tablet having an ovoid-rectangular shape, it is preferred that the tablets have an overall dimension of 0.400±0.05 inches by 0.325±0.05 inches with a thickness of 0.240±0.025 inches (biconvex faces) or 0.195±0.025 inches (flat faces).

In another embodiment of the present invention the overall tablet weight ranges from 350 to 450 mg, preferably from 375 to 425 mg, more preferably from 385 to 410 mg. Further, within the context of the present invention, the tablet weight range of 350 to 450 mg, preferably from 375 to 425 mg, more preferably from 385 to 410 mg correspond to the total weight of the unit dose form.

Therefore, the pharmaceutical compositions (i.e., extended-release oral dosage form of gepirone) of the present invention preferably contain between about 55 to 100 mg (preferably from to 80 mg, more preferably 60 or 80 mg) of the active ingredient per unit dose.

The tablets may, if desired, be coated using known methods and excipients that may include enteric coating using for example hydroxypropylmethylcellulose phthalate. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

### Treatment Methods:

Gepirone can be orally administered in once-a-day extended release dosage forms, which contain gepirone hydrochloride (or a bioactive metabolite thereof), a cellulosic polymer matrix and suitable amounts of pharmaceutical excipients. The resultant gepirone formulation yields oral products the take about 19 to about 24 hours to release 90 to 95% of the active agent.

The gepirone extended-release composition and dosage forms of the invention are designed to deliver a therapeutically effective amount of gepirone or a pharmaceutically acceptable salt thereof to a mammal, preferably a human patient.

### Ipsapirone

Ipsapirone (also known as 2-(4-(4-(2-pyrimidinyl)-1-piperazinyl)-butyl) 1,2-benzoisothiazol-3(2H)-one 1,1-dioxide hydrochloride), is an azapirone 5-HT_{1A} partial agonist.

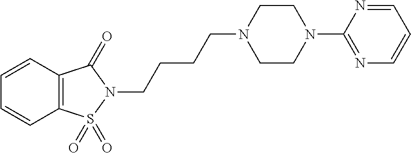 [SMILES: O=C1c2ccccc2S(=O)(=O)N1CCCCN1CCN(c2ncccn2)CC1]

The drug has been shown effective in the treatment of anxiety disorders, such as generalized anxiety disorder (GAD), panic disorder, and obsessive compulsive disorder (Glitz. D. A., Pohl R., Drugs 1991, 41:1 1; Cadieux, Amer. Family Physician 1996 53: 2349-2353). Ipsapirone and methods of making ipsapirone are disclosed in U.S. Pat. No. 4,988,700 and Traber et al (Trends Pharmacol Sci 1987; 8: 432-7), both of which are incorporated herein by reference.

### Tandospirone

Tandospirone (also known as N-[4-[4-(2-pyrimidinyl)-1-piperazinyl]butyl]-2,3-norbornanedicarboximide), is an azapirone 5-HT_{1A} partial agonist.

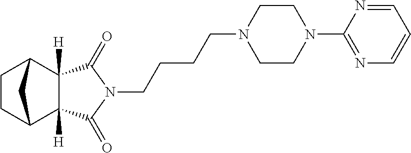 [SMILES: O=C1[C@@H]2[C@@H]3CC[C@@H](C3)[C@@H]2C(=O)N1CCCCN1CCN(c2ncccn2)CC1]

Tandospirone (N-[4-[4-(2-pyrimidinyl)-1-piperazinyl]butyl]-2,3-norbornanedicarboximide) can be obtained by the process according to in U.S. Pat. Nos. 4,507,303; 4,543,355; 4,598,078 and 5,011,841, all of which are incorporated herein by reference.

### Adatanserin

Adatanserin (also known as (N-[2-[4-(2-pyrimidinyl)-1-piperazinyl]ethyl]tricyclo[3.3.1.1^{3,7}]decane-1-carboxamide) is also an azapirone 5HT_{1A} partial agonist but also has activity as an 5HT₂ antagonist.

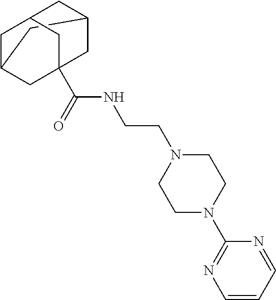 [SMILES: O=C(NCCN1CCN(c2ncccn2)CC1)C12CC3CC(CC(C3)C1)C2]

Adatanserin can be formed and administered according to the disclosures the following references incorporated herein by reference (U.S. Pat. No. 5,380,725, US 2005/0095286 and US 2009/0281112). Clinical studied have shown activity in the treatment of attention deficit disorder, anxiety, depression, sexual dysfunction and other disorders (U.S. 2009/0281112, the text of which is incorporated herein by reference).

3-OH-Adatanserin is a major metabolite of adatanserin. 3-OH-adatanserin is also a partial agonist of 5-HT_{1A}.

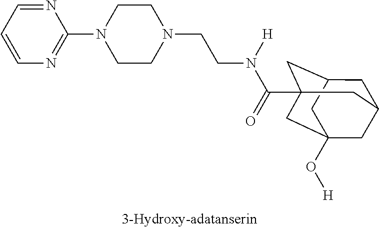 [SMILES: O=C(NCCN1CCN(c2ncccn2)CC1)C12CC3CC(CC(O)(C3)C1)C2]

Clinical studied have shown activity in the treatment of attention deficit disorder, anxiety, depression, sexual dysfunction and other disorders (U.S. 2009/0281112, the text of which is incorporated herein by reference). 3-OH-adatanserin has the same chemical structure as adatanserin except for a hydroxyl group in the 3 position.

### Flesinoxan

Flesinoxan (also known as (+)-N-[2-[4-(2,3-dihydro-2-hydroxymethyl-1,4-benzodioxin-5-yl)-lpiperaxinyl]ethyl]-4-fluorobenzamide hydrochloride) is a high affinity and high selectivity 5-HT_{1A} receptor agonist.

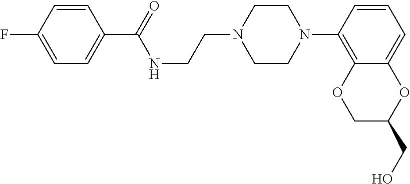 [SMILES: O=C(NCCN1CCN(c2cccc3c2OC[C@H](CO)O3)CC1)c1ccc(F)cc1]

Flesinoxan has been shown to have both anxiolytic and antidepressant activity (Hadrava et al, Neuropharmacology, 1995; 34 (10): 1311-1326).

### Buspirone

Buspirone (also known as (8-[4-4-pyrimidin-2-ylpiperazin-1-yl)butyl]-8-azaspiro[4.5]decane-7,9-dione) is an azapirone 5-HT_{1A} partial agonist.

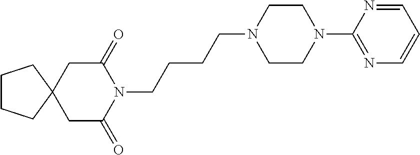 [SMILES: O=C1CC2(CCCC2)CC(=O)N1CCCCN1CCN(c2ncccn2)CC1]

The drug has been shown effective in the treatment of anxiety disorders, such as generalized anxiety disorder (GAD), panic disorder, and obsessive compulsive disorder (Glitz. D. A., Pohl R., Drugs 1991, 41:1 1; Cadieux, Amer. Family Physician 1996 53: 2349-2353).

Disorders

The following disorders often coincide with elevated cholesterol levels. Furthermore, as shown in Example 5 below, the inventors have discovered that the efficacy of the above 5HT1A agonists is increased when cholesterol levels are elevated. As such, another embodiment of the present invention covers a method of treating one of the below disorders comprising administering a therapeutically effective amount of at least one 5-HT_{1A} receptor agonist to a subject with elevated cholesterol levels in need thereof. The disorders include attention deficit disorder, depression, sexual dysfunction, psychoses, aggressive behavior, alcohol dependence, obesity, anxiety, drug abuse/addiction, sleep disorders, and behavioral/cognitive symptoms of Alzheimer's.

### Attention Deficit Disorder

Attention deficit disorder (ADD) is a learning disorder that relates to developmentally inappropriate inattention and impulsivity. ADD may also be referred to as disruptive behavior disorder or minimal brain dysfunction. ADD may be present with or without hyperactivity. A common disorder, ADD accounts for more child mental health referrals than any other single disorder. ADD is estimated to affect 3 to 50 of school-aged children, and is much more frequent in males than in females, with a male-to-female ratios ranging from 4:1 to 9:1. On the average, at least one child in every classroom in the United States needs help for the disorder. ADD often continues into adolescence and adulthood, and can cause a lifetime of frustrated dreams and emotional pain. In addition, ADD may affect the behavior of children at any cognitive level.

ADD is a diagnosis applied to children and adults who consistently display certain characteristic behaviors over a period of time. The most common behaviors fall into two categories: inattention and impulsivity. Attention deficit disorder with hyperactivity is diagnosed when the signs of overactivity are obvious. Inappropriate inattention causes increased rates of activity and impersistence or reluctance to participate or respond. A subject suffering from ADD exhibits a consistent pattern of inattention and/or hyperactivity-impulsivity that is more frequent and severe than is typically observed in individuals at a comparable level of development. Such subjects must suffer clear evidence of interference with developmentally appropriate social, academic, or occupational functioning.

Although subjects with ADD and without hyperactivity may not manifest high activity levels, most exhibit restlessness or jitteriness, short attention span, and poor impulse control. These are qualitatively different from those seen in conduct and anxiety disorders. Inattention is described as a failure to finish tasks started, easy distractibility, seeming lack of attention, and difficulty concentrating on tasks requiring sustained attention. Impulsivity is described as acting before thinking, difficulty taking turns, problems organizing work, and constant shifting from one activity to another. Impulsive responses are especially likely when involved with uncertainty and the need to attend carefully. Hyperactivity is featured as difficulty staying seated and sitting still, and running or climbing excessively. A more complete description of the symptoms and diagnostic criteria of attention deficit disorder with or without hyperactivity are provided by DSM-IV (Diagnostic and Statistical Manual of Mental Disorders, 1994; 78-85), which is incorporated herein by reference.

No single treatment has been completely effective for attention deficit disorder. Psychostimulant medications combined with behavioral and cognitive therapies (e.g., selfrecording, self-monitoring, modeling, and role-playing) have the greatest controlling influence on symptom expression. Used alone, medication has been effective predominantly with less aggressive ADD children coming from stable home environments. Elimination diets, megavitamin treatments, psychotherapy, and biochemical interventions (e.g., the administration of neurochemicals) have had only minor, unsustained effects.

For decades, medications have been used to treat the symptoms of ADD. The three most common medications in both adults and children are the stimulants; methylphenidate (RITALIN™), dextroamphetamine (DEXEDRINE™ or DEXTROSTAT™), and pemoline (CYLERT™). For many people, these medicines dramatically reduce their hyperactivity and improve their ability to focus, work, and learn. The medications may also improve physical coordination, such as handwriting and ability in sports. Recent research by National Institute of Mental Health suggests that these medicines may also help children with an accompanying conduct disorder to control their impulsive, destructive behaviors. Nine out of 10 children improve on one of these three stimulant drugs.

Different doctors use the medications in slightly different ways. CYLERT™ is available in one form, which naturally lasts 5 to 10 hours. RITALIN™ and DEXEDRINE™ come in short-term tablets that last about 3 hours, as well as longer-term preparations that last through the school day.

Stimulant drugs, when used with medical supervision, are usually considered safe. However, a common problem with stimulant drugs is that they can be addictive to teenagers and adults if misused. While on these medications, some children may lose weight, have less appetite, and temporarily grow more slowly. Others may have problems falling asleep. Some doctors believe that stimulants may also make the symptoms of Tourette's syndrome worse.

The most commonly prescribed ADD medication is RITALIN™, which is generally more effective than tricyclic antidepressants (e.g., IMIPRAMINE™), caffeine, and other psychostimulants (e.g., PEMOLINE™ and DEANOL™) and has fewer side effects than dextroamphetamine. Common side effects of RITALIN™ are sleep disturbances (e.g., insonmia), depression or sadness, headache, stomachache, suppression of appetite, elevated blood pressure, and, with large continuous doses, a reduction of growth. Long-term benefits of medication with RITALIN™, however, have not been demonstrated conclusively. Some research indicates that use of medication permits participation in activities previously inaccessible because of poor attention and impulsivity. The frequency of side effects, potential addictiveness, and limited success of stimulant drugs has led to a search for alternate means of treating or preventing attention deficit disorders.

However, agonism of the 5-HT_{1A} receptor has been correlated with treatment of attention disorders as described in the following references which are incorporated herein by reference (U.S. Pat. No. 4,423,049, U.S. Pat. No. 4,771,053, U.S. Pat. No. 5,106,849, and Bison, Journal of Clinical Psychopharma, 1990; 10: S2-S5). Furthermore, 5HT_{1A} agonists have shown effective in treating ADD (US 2009/012112, US 2009/028111) also incorporated herein by reference).

### Depression

Depression is a psychiatric disorder, characterized by a pervasive low mood, loss of interest in usual activities and diminished ability to experience pleasure. Clinical depression affects about 8-17% of the population on at least one occasion in their lives, before the age of forty. In the United States, it has been estimated that approximately 14 million adults suffer depression per year. Although the term “depression” is commonly used to describe a temporary condition when one “feels blue”, clinical depression is a serious and often disabling condition that can significantly affect a person's work, family and school life, sleeping and eating habits, general health and ability to enjoy life. The course of clinical depression varies widely. Depression can be a once in a life-time event or have multiple recurrences. It can appear either gradually or suddenly, and either last for a few months or be a life-long disorder. Depression is a major risk factor for suicide, and people with depression suffer from higher mortality due to other causes of death.

Clinical depression may be a primary condition, or it may be secondary to another condition, such as chronic pain. Clinical depression can present with a variety of symptoms. Most patients display a marked change in mood, a deep feeling of sadness, and a noticeable loss of interest or pleasure in favorite activities. Other symptoms include persistent sadness or anxiety, loss of appetite and/or weight loss, increased appetite and overeating and weight gain, insomnia, restlessness or irritability. The depressed patient may experience feelings of worthlessness, inappropriate guilt, helplessness, hopelessness, and/or pessimism. He may have difficulty thinking, concentrating, remembering or making decisions. The depressed patient may have suicidal thoughts, and may attempt suicide. Decreased energy, fatigue, and sluggishness, along with persistent physical symptoms that do not respond to treatment, such as headaches, digestive problems, and chronic pain, are often experienced by depressed patients. Diagnosis of depression is detailed in Diagnostic and Statistical Manual of Mental Disorders (Fourth Ed.) (American Psychiatric Association (1994), herein incorporated by reference in its entirety.

Genetic factors and environmental factors, such as prolonged stress at home or work, coping with the loss of a loved one, or traumatic events, may also contribute to depression.

The depression referred to in the present application can include but is not limited to; major depressive disorder (also known as recurrent depressive disorder, clinical depression, major depression, unipolar depression, or unipolar disorder), melancholic depression, atypical depression, catatonic depression, postpartum depression, mental and behavioral disorders associated with the puerperium, and seasonal affective disorders.

Major Depression and disorders associated therewith have been linked to early death due to cardiovascular mechanisms secondary to increased lipids and/or the metabolic syndrome. Psychiatric drugs have also been implicated in deleterious effects of weight, lipids and glucose. The typical treatment for Major Depression is the Selective Serotonin Reuptake Inhibitors (SSRIs). Several studies have shown and the data in this application confirms that fluoxetine and sertraline elevate cholesterol levels.

Furthermore, although the literature suggests that cholesterol levels were low in patients with major depression, depressed patients typically die 10-20 years earlier than non depressed patients largely due to cardiovascular disease. Therefore, the inventors of this invention questioned this literature and investigated total cholesterol levels in 10 studies of gepirone at baseline before any treatment (See Examples 1-2). It was surprisingly discovered that in this population of depressed subjects cholesterol levels were increased.

The question of the impact of cholesterol on drug efficacy has also been investigated. With currently available antidepressant drugs, predominately SSRIs and SNRIs, only approximately one third of patients with major depression experience an adequate response. The reason for this is not known. There have been several attempts to try to understand which population will respond to antidepressants and which populations will not. However, no such studies have been don't on 5-HT_{1A} agonists.

The present inventors further studied the effect of 5-HT_{1A} agonists on cholesterol levels and it was surprisingly found that the 5-HT_{1A} agonists decreased cholesterol levels in a clinically significant amount. This was not expected in view of the above studies of SSRIs. Specifically, as noted above in patients with major depression, treatment with SSRI's results in increased cholesterol levels, whereas the present invention is based, in part, on the discovery that treatment with 5HT_{1A} agonists results in statistically significant decreases in cholesterol levels (See Examples 3-4).

5HT_{1A} agonists are essentially free of the adverse events imparted by statins other than limited mild dizziness and nausea. 5HT_{1A} agonists are not known to inhibit HMG-CoA reductase, nor do they have chemical structures that are similar to statins. While not wishing to be bound by any theory, it is theorized that 5HT_{1A} agonists reduce cholesterol by another mechanism than statins.

The lowering effect of 5HT_{1A} agonists on lipid levels will improve quality and duration of life in depressed patients.

Furthermore, 5HT_{1A} agonists have shown effective in treating depression (U.S. application Ser. No. 13/709,986 the texts of which are incorporated herein by reference).

### Sexual Dysfunction

Sexual Dysfunction may be defined as difficulty during any stage of the sexual act (which includes desire, arousal, orgasm, and resolution) that prevents the individual or couple from enjoying sexual activity. Sexual dysfunction disorders are generally classified into four categories: sexual desire disorders, sexual arousal disorders, orgasm disorders, and sexual pain disorders.

Sexual desire disorders (decreased libido) may be caused by a decrease in the normal production of estrogen (in women) or testosterone (in both men and women). Other causes may be aging, fatigue, pregnancy, and medications (such as anti-depressants such as fluoxetine, sertraline, and paroxetine are well known for reducing desire in both men and women. Psychiatric conditions, such as depression and anxiety, can also cause decreased libido.

Sexual arousal disorders were previously known as frigidity in women and impotence in men. These have now been replaced with less judgmental terms. Impotence is now known as erectile dysfunction, and frigidity is now described as any of several specific problems with desire, arousal, or anxiety. For both men and women, these conditions may appear as an aversion to, and avoidance of, sexual contact with a partner. In men, there may be partial or complete failure to attain or maintain an erection, or a lack of sexual excitement and pleasure in sexual activity.

Orgasm disorders are a persistent delay or absence of orgasm following a normal sexual excitement phase. The disorder occurs in both women and men. Again, the SSRI antidepressants are frequent culprits—these may delay the achievement of orgasm or eliminate it entirely.

Sexual pain disorders affect women almost exclusively, and are known as dyspareunia (painful intercourse) and vaginismus (an involuntary spasm of the muscles of the vaginal wall, which interferes with intercourse). Dyspareunia may be caused by insufficient lubrication (vaginal dryness) in women.

Sexual dysfunctions are more common in the early adult years, with the majority of people seeking care for such conditions during their late 20s through 30s. The incidence increases again in the geriatric population, typically with gradual onset of symptoms that are associated most commonly with medical causes of sexual dysfunction. Sexual dysfunction is more common in people who abuse alcohol and drugs. It is also more likely in people suffering from diabetes and degenerative neurological disorders. Ongoing psychological problems, difficulty maintaining relationships, or chronic disharmony with the current sexual partner may also interfere with sexual function.

Symptoms of sexual dysfunction may include loss of libido, inability to feel aroused, painful intercourse in both male and female patients. In men, symptoms may include inability to attain or maintain an erection, delay or absence of ejaculation, and inability to control timing of ejaculation. In women, symptoms may include inability to relax vaginal muscles enough to allow intercourse, inadequate vaginal lubrication before and during intercourse, inability to attain orgasm, and burning pain on the vulva or in the vagina with contact to those areas.

Sexual dysfunction is common among individuals with depression. Depressed individuals show decreased sexual interest and reported reduced levels of arousal. Sexual dysfunction is also a common side effect of antidepressant treatment, particularly pharmacotherapy with serotonin reuptake inhibitors (SRIs). The sexual response cycle consists of 4 phases: desire, arousal, orgasm, and resolution. All of these phases may be affected by reproductive hormones and neurotransmitters. Estrogen, testosterone, and progesterone promote sexual desire. Dopamine promotes desire and arousal. Norepinephrine promotes arousal. Prolactin inhibits arousal. Oxytocin promotes orgasm. Serotonin may have a negative impact on the desire and arousal phases of the sexual response cycle, possibly due to its inhibition of dopamine and norepinephrine.

Treatment of sexual dysfunction involves identifying the specific cause and, often, treating the underlying condition. Medical causes that are reversible or treatable are usually managed medically or surgically. Physical therapy and mechanical aides may prove helpful for some people experiencing sexual dysfunction due to physical illnesses, conditions, or disabilities.

5HT_{1A} agonists have shown effective in treating sexual dysfunction (US 2009/0281112, and U.S. Pat. No. 7,538,116, the texts of which are incorporated herein by reference).

### Psychoses

Psychosis is a general term for a mental state described as involving a loss of contact with reality. People experiencing psychoses may report hallucinations or delusional beliefs, and may exhibit personality changes and disorganized thinking. This may be accompanied by unusual or bizarre behaviour, as well as difficulty with social interaction and impairment in carrying out the activities of daily living.

The signs and symptoms of psychoses include one or more of hallucinations, delusions, thought disorder, and lack of insight. Hallucinations are defined as sensory perception in the absence of external stimuli. Hallucinations may occur in any of the five senses and take on almost any form, which may include simple sensations (such as lights, colors, tastes, and smells) to more meaningful experiences such as seeing and interacting with fully formed animals and people, hearing voices and complex tactile sensations. Auditory hallucinations, particularly the experience of hearing voices, are a common and often prominent feature of psychosis. Delusions are typically beliefs which are sometimes paranoid in nature. Psychotic delusions have been classified as primary or secondary. Primary delusions are defined as having no known source and not being comprehensible in terms of normal mental processes. Secondary delusions may be understood as being influenced by the person's background or situation, such as ethnicity, sexuality, religion, or superstition. Thought disorder is an underlying disturbance to conscious thought. It is classified largely by its effects on speech and writing, with affected persons often showing speech pressures (speaking incessantly and quickly), derailment or flight of ideas (switching topic mid-sentence or inappropriately), thought blocking, and rhyming or punning. Lack of insight is generally defined as the psychotics lack of awareness of the unusual, strange, or bizarre nature of his experience or behavior.

Psychoses are classified descriptively based on behavioral and clinical observations. This approach is adopted in the standard guide to psychiatric diagnoses employed in the United States, the Diagnostic and Statistical Manual of Mental Disorders (DSM), incorporated herein by reference. The DSM lists nine psychoses, or formal psychotic disorders. The formal psychotic disorders are: schizophrenia, schizoaffective disorder, schizophreniform disorder, brief psychotic disorder, delusional, shared psychotic disorder, substance induced disorder, psychosis due to a general medical condition, and psychosis—not otherwise specified.

The DSM classifies psychotic disorders as traditional psychotic illnesses, psychosis due to general medical conditions, and substance induced psychosis. Traditional psychoses may be related to schizophrenia, bipolar disorder (manic depression), severe clinical depression, severe psychosocial stress, and sleep deprivation. Psychosis arising from “organic” (non-psychological) can be associated with the neurological disorders, electrolyte disorders, hypoglycemia, lupus, AIDS, leprosy, malaria, and other conditions. Neurological disorders that can give rise to psychoses may include brain tumours, multiple sclerosis, sarcoidosis, lyme disease, syphilis, Alzheimer's Disease, and Parkinson's Disease. Electrolyte disorders may include hypocalcemia, hypematremia, hyponatremia, hypokalemia, hypomagnesemia, hypermagnesemia, hypercalcemia, and hypophosphatemia. Psychoses may also be caused by ingestion of many legal and illegal substances, including alcohol, dextromethorphan, certain antihistamines, cold medications including phenylpropanolamine, barbiturates, benzodiazepines, isotretinoin, anticholinergic drugs (such as atropine, scopolamine, and Jimson weed), antidepressants, L-dopa, antiepileptics, stimulants (including cocaine, amphetamines, methamphetamine, methylphenidate, MDMA (ecstasy)) hallucinogens (including cannabis, psilocybin, mescaline, and PCP), psychotropics (including LSD).

5HT_{1A} agonists have shown effective in treating psychoses (US 2009/0281112, and U.S. Pat. No. 6,534,507, the texts of which are incorporated herein by reference).

### Aggressive Behavior

Aggressive behavior is behavior that is intended to cause harm or pain. It can be either physical or verbal. To distinguish, behavior that accidentally causes harm or pain is not aggression, and aggression is not synonymous with assertiveness. Aggression is currently categorized broadly as either (1) hostile, affective, or retaliatory aggression, or as (2) instrumental, predatory, or goal-oriented aggression. Some evidence suggests that the two types are psychologically and physiologically different. For example, some research indicates that people with tendencies toward affective aggression have lower IQs than those with tendencies toward predatory aggression. There are also thought to be gender-based differences.

The biology of aggressive behavior is complex. Aggression is directed to and often originates from outside stimuli, but has a very distinct internal character. Within the brain, the amygdala has been shown to be critically involved in aggression, as stimulation of the amygdala results in augmented aggressive behavior. Further, lesions of this area greatly reduce aggression. In addition, the hypothalamus serves a regulatory role in aggression. The hypothalamus has been shown to cause aggressive behavior when electrically stimulated and expresses receptors that help determine aggression levels based on their interactions with the neurotransmitters serotonin and vasopressin. The prefrontal cortex is also associated with the regulation of aggression. Reduced activity of the prefrontal cortex, in particular its medial and orbitofrontal portions, has been associated with violent/antisocial aggression. Specifically, regulation of the levels of the neurotransmitter serotonin in the prefrontal complex has been connected with a particular type of pathological aggression.

Clinical and preclinical evidence supports 5-HT_{1A} partial agonists for use in treating depression as well as impulse control disorders and alcohol abuse (van Hest, Psychopharm., 107: 474 (1992); Schipper et al, Human Psychopharm., 6: S53 (1991); Cervo et al, Eur. J. Pharm., 158: 53 (1988); Glitz, D. A., Pohl, R., Drugs, 41: 11 (1991)). 5HT_{1A} agonists have shown effective in treating aggression (US 2009/0281112, and U.S. Pat. No. 6,534,507, the texts of which are incorporated herein by reference).

### Alcohol/Drug Dependence

Alcohol Dependence, or alcoholism, and drug dependence is a condition characterized by the harmful consequences of repeated alcohol or drug use, a pattern of compulsive alcohol or drug use, and physiological dependence on alcohol or drugs. The physiological dependence is characterized by development of tolerance to increased levels of alcohol or drugs and symptoms of withdrawal upon discontinuation of abuse. Alcohol or drug dependence is diagnosed when these behaviors become persistent, disabling, or distressing.

Persons suffering from alcoholism or drug addiction face many complications. School and job performance may suffer. Child care or household responsibilities may be neglected. Absences from school or job may occur. The individual may use alcohol or drugs in physically hazardous circumstances, such as driving or operating machinery while intoxicated. Moreover, legal difficulties may arise because of alcohol or drug use, including arrests for intoxicated behavior or for drunk driving. Alcohol and drug intoxication causes significant intellectual impairment.

Physical problems seen with alcohol abuse may include depression, blackouts, liver disease, and other complications. Nearly every organ system is effected. Gastrointestinal effects include gastritis, stomach or duodenal ulcers, liver cirrhosis and pancreatitis. There is also an increased rate of cancer of the esophagus, stomach, and other parts of the gastrointestinal tract. Cardiovascular effects include hypertension, along with an elevated risk of heart disease. Nervous system effects include peripheral neuropathy, as evidenced by muscular weakness, paresthesis, and decreased peripheral sensation. Central nervous system effects include cognitive deficits, severe memory impairment, and degenerative changes in the cerebellum, resulting in poor balance and coordination. Individuals with this disorder are at increased risk for accidents, violence, and suicide. Severe Alcohol Intoxication also contributes to disinhibition and feelings of sadness and irritability, which contribute to suicide attempts and completed suicides.

Individuals suffering from alcoholism or drug addiction continue to abuse alcohol or drugs despite knowing that doing so causes significant social or interpersonal problems for them. Alcoholics and drug addicts may devote substantial time, effort, and financial resources to obtaining and consuming alcohol or drugs.

Individuals with Alcohol Dependence or drug dependence, including pharmaceuticals, are at increased risk for Major Depressive Disorder, other Substance-Related Disorders (e.g., drug addiction), Conduct Disorder in adolescents, Antisocial and Emotionally Unstable (Borderline) Personality Disorders, Schizophrenia, and Bipolar Disorder.

Clinical and preclinical evidence supports 5-HT_{1A} partial agonists for use in treating depression as well as impulse control disorders and alcohol abuse (van Hest, Psychopharm., 107: 474 (1992); Schipper et al, Human Psychopharm., 6: S53 (1991); Cervo et al, Eur. J. Pharm., 158: 53 (1988); Glitz, D. A., Pohl, R., Drugs, 41: 11 (1991)). 5HT_{1A} agonists have shown effective in treating alcohol and drug (substance) abuse (US 2009/0281112, and U.S. Pat. No. 6,534,507, the texts of which are incorporated herein by reference).

### Obesity

Obesity is a condition in which the natural energy reserve, stored in the fatty tissue of humans and other mammals, exceeds healthy limits. It is commonly defined as a body mass index (weight divided by height squared) of 30 kg/m² or higher. Although obesity is an individual clinical condition, some authorities view it as a serious and growing public health problem. Some studies show that excessive body weight has been shown to predispose to various diseases, particularly cardiovascular diseases, diabetes mellitus type 2, sleep apnea and osteoarthritis.

Obesity can be classified in absolute or relative terms using measurements such as body mass index (BMI). BMI is calculated with the following formula:

Metric:BMI=kg/m²

where kg is the subject's weight in kilograms and m is the subject's height in meters. The BMI can be used to classify, as follows, using definitions established by the World Health Organization:
A BMI less than 18.5 is underweight
A BMI of 18.5-24.9 is normal weight
A BMI of 25.0-29.9 is overweight
A BMI of 30.0-39.9 is obese
A BMI of 40.0 or higher is severely (or morbidly) obese
A BMI of 35.0 or higher in the presence of at least one other significant comorbidity is also classified by some bodies as morbid obesity.
Waist: Hip ratios, waist circumference, and body fat measurement are also used to assess obesity.

A large number of medical conditions have been associated with obesity. Health consequences include those associated with increased fat mass (osteoarthritis, obstructive sleep apnea, social stigma) or increased number of fat cells (diabetes, cancer, cardiovascular disease, non-alcoholic fatty liver disease). Mortality is increased in obesity, with a BMI of over 32 being associated with a doubled risk of death. Moreover, alterations in the body's response to insulin), a proinflammatory state, and an increased tendency to thrombosis (prothrombotic state), are seen in obese patients.

There remains a need for more effective therapy for weight loss. 5HT_{1A} agonists have shown effective in treating obesity and related disorders (US 2009/0281112, the text of which is incorporated herein by reference).

### Anxiety

Anxiety is a condition characterized by cognitive, physical, emotional, and behavioral components, combining to result in feelings typically recognized as fear, apprehension, or worry. Anxiety is often accompanied by physical sensations such as heart palpitations, nausea, chest pain, shortness of breath, stomach aches, or headache. The cognitive component entails recognition of a threat of certain danger. Physically, the body prepares the organism to deal with such a threat, including increased blood pressure, heart rate, perspiration, and blood circulation to major muscle groups. The body also experiences inhibition of the immune and digestive system functions. External signs of anxiety may include pale skin, sweating, trembling, or pupillary dilation. Emotionally, anxiety causes a sense of dread or panic and physically causes nausea, diarrhea, and chills. Behaviorally, both voluntary and involuntary behaviors may arise directed at escaping or avoiding the source of anxiety. Anxiety is not always pathological. Rather, it is a common emotion along with fear, anger, sadness, and happiness, with an important function in relation to survival. However, anxiety can be maladaptive, with the most extreme cases being expressed as anxiety disorders.

Anxiety can be classified by its severity. Severe anxiety disorders only affects a small minority of anxiety sufferers, and may include severe panic and anxiety disorders like obsessive-compulsive disorder (OCD), post-traumatic stress disorder (PTSD), social phobias, and stress disorders. Severe anxiety disorders are highly treatable but require medical diagnosis. Generalized anxiety disorder, characterized as by mild to moderate anxiety, is far more common but harder to identify. Generalized anxiety disorder is characterized by compulsive worrying and physical symptoms of anxiety which persist for more than six months.

Although acute attacks of anxiety are not experienced by every anxiety sufferer, they are a common symptom. Anxiety attacks usually come without warning, and although the fear is generally irrational, the perceived danger is very real. A person experiencing an anxiety attack will often feel as if they are about to die or pass out, may experience fear, and may feel the need to avoid certain stressful situations or social situations due to fear of embarrassment. The person may experience considerable confusion and irritability when experiencing anxiety. Physical symptoms include hot flushes, chest pain, sudden exhaustion, headaches, and shortness of breath, digestive problems, and nausea.

Anxiety may be treated with medication, therapy, or a combination of both. Other treatment modalities may include self-help techniques, lifestyle changes, and alternative medicine for relief and wellness. Medications used for treatment of anxiety include antidepressants. These may include selective serotonin reuptake inhibitors, tricyclic antidepressants, and monoamine oxidase inhibitors. Other antianxiety drugs include benzodiazepines and buspirone. While these may be effective, the side effects of such treatments can be significant, including the development of dependency or addiction. The frequency of side effects, potential addictiveness, and limited success of stimulant drugs has led to a search for alternate means of treating or preventing attention deficit disorders.

However, agonism of the 5-HT_{1A} receptor has been correlated with treatment of anxiety as described in the following references which are incorporated herein by reference (U.S. Pat. No. 4,423,049, U.S. Pat. No. 4,771,053, U.S. Pat. No. 5,106,849, and Bison, Journal of Clinical Psychopharma, 1990; 10: S2-S5). Furthermore, 5HT_{1A} agonists have shown effective in treating anxiety (US 2009/0281112, and U.S. Pat. No. 6,534,507 incorporated herein by reference).

### Sleep Disorders

Sleep disorders are a disorder of the sleep patters of a person or animal. These disorders can be serious enough to interfere with normal physical, mental and emotional functioning. Sleep disorder can include but are not limited to: insomnia, bruxism (involuntary grinding or clenching of teeth while sleepting), delayed sleep phase syndrome (inability to awaken or fall asleep at socially acceptable times), hypopnea syndrome (abnormally shallow breathing or slow respiratory rate while sleeping), narcolepsy (excessive daytime sleepiness), cataplexy (a sudden weakness in the motor muscles that can result in collapse on the floor), night terrors, parasomnias (disruptive sleep-related events involving inappropriate actions during sleep), and sleep apnea. Sleep disorders are often a resulting symptom of depression, anxiety, or other mental health disorders.

However, agonism of the 5-HT_{1A} receptor has been correlated with treatment of sleep disorders (U.S. Pat. No. 4,423,049, U.S. Pat. No. 4,771,053, U.S. Pat. No. 5,106,849, and Bison, Journal of Clinical Psychopharma, 1990; 10: S2-S5). Furthermore, 5HT_{1A} agonists have shown effective in treating sleep disorders (US 2009/0281112 incorporated herein by reference).

### Behavioral/Cognitive Symptoms of Alzheimer's

Alzheimer's disease (AD) is the most common form of dementia. There is no cure for the disease which worsens as it progresses and eventually leads to death. Alzheimer's is predicted to affect 1 in 85 people globally by 2050.

Although Alzheimer's disease develops differently for every individual, there are many common symptoms such as, difficulty remembering recent events, confusion, irritability, aggression, mood swings, trouble with language and long-term memory loss. Suffers also typically withdraw from family and society, and eventually bodily functions are lost leading to death. The average life expectancy following Alzheimer's diagnosis is approximately seven years.

Agonism of the 5-HT_{1A} receptor has been correlated with treatment of cognitive symptoms of Alzheimer's disease (U.S. Pat. No. 4,423,049, U.S. Pat. No. 4,771,053, U.S. Pat. No. 5,106,849, and Bison, Journal of Clinical Psychopharma, 1990; 10: S2-S5). Furthermore, 5HT_{1A} agonists have shown effective in treating cognitive symptoms of Alzheimer's disease (US 2009/0281112 incorporated herein by reference).

Having generally described this invention, a further understanding can be obtained by reference to certain specific examples, which are provided herein for purposes of illustration only, and are not intended to be limiting unless otherwise specified.

### EXAMPLES

### Example 1

### Evaluation of the Effect of SSRIs and Gepirone-ER on Cholesterol Levels in Depressed Subjects

Methods:

In 10 clinical studies of the antidepressant effects of Gepirone-ER comprised of 2302 subjects with Major Depression safety measurements included non-fasting measurements of cholesterol at baseline and at 8 weeks (endpoint). A JMP-7 one way ANOVA of the data provided results for individual studies which were then analyzed by meta-analysis.

Results:

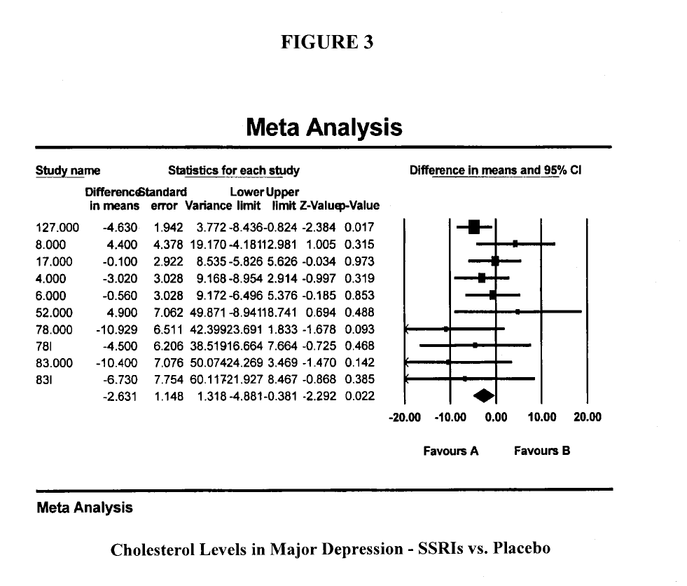

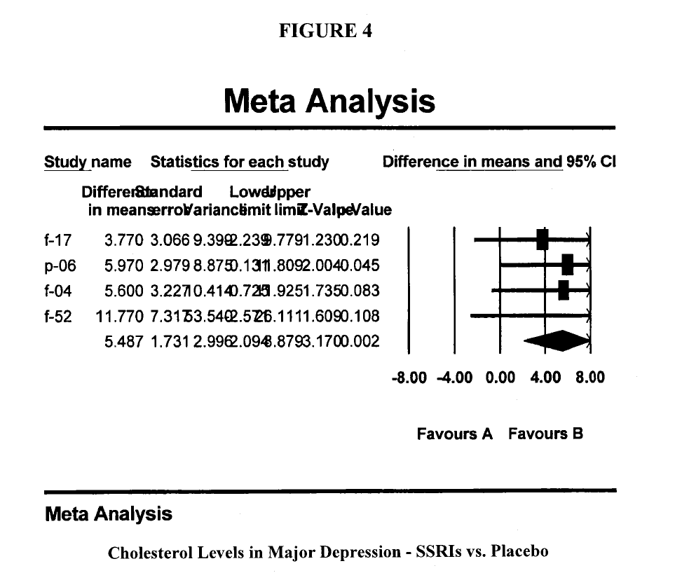

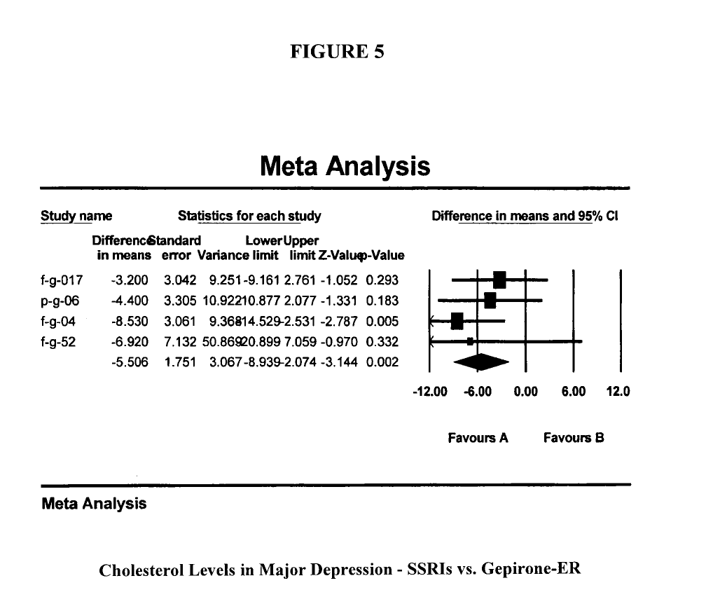

In 10 studies (n=931) gepirone-ER treated patients had numerically reduced cholesterol levels in 8 studies. A meta-analysis of the ten studies shows gepirone-ER statistically significantly better then placebo (n=938) p=0.022 (See FIG. 3). One study of paroxetine (n=143) showed placebo statistically better than paroxetine p=0.045, and three studies of flyoxetine (n=295) resulted in placebo statistically better then fluoxetine p=0.014 (See FIG. 4). In the 4 SSRI studies, gepirone-ER was statistically better than the SSRI p=0.002 (See FIG. 5).

In patients with Major Depression, treatment with SSRI's results in increased cholesterol levels, whereas treatment with gepirone-ER results in statistically significant decreases in cholesterol levels. The lowering effect of gepirone-ER on lipid levels should improve quality and duration of life in depressed patients.

### Example 2

### Evaluating Total Cholesterol Level in Untreated Subjects with Major Depression

Literature suggests that lipid levels in depressed patients are reduced. However, depressed patients die on average 10-20 years earlier than non-depressed patients largely due to cardiovascular disease. Generally, depressed subjects don't exercise and have poor diets. These facts suggest a reevaluation of lipid levels in this population.

Methods:

In 10 studies, 2564 depressed subjects classic (MDD): (loss of appetite, insomnia, n=1719) and atypical (ADD)(hyperphagia, hypersomnia, n=845), selected for participation in trials of new antidepressants were evaluated pre-treatment. The subjects were recruited from 55 sites across the United States from 1991 2003. The average age was 38.4±11 years and 51.5% were female. Subjects with medical illnesses including: diabetes or cardiovascular diseases were excluded. Concomitant medications were restricted. Subjects taking statins were removed. Initial screening included total cholesterol levels, weight and height. Depression severity was approximately ⅓ each: mild, moderate and severe. The results were compared with the National Health and Nutrition Examination Survey (NHANES) data base. Three studies done from 1991-1992 were compared to the NHANES III data, and the remaining studies done from 1999 to 2003 were compared to the NHANES 1999-2006 data.

Results:

For all subjects, total cholesterol values were elevated in 6 of 10 studies of major depression compared to NHANES data. The average increase amounted to 1.2 mg/dL, NS; ADD subset +3.3, NS. Male depressed subjects were increased by 4.7 mg/dL p=0.0047; ADD subset +8.4, p=0.059. Female depressed subjects had a 0.57 mg/dL decrease in cholesterol levels NS: ADD subset +0.9, NS. Compared by age group, depressed subjects had increased cholesterol levels: 20-39, +2.1 NS; 40-59+5.1, p=0.016, and 60-74, +20.1 p=0.014. BMI for classic major depression was 29.5 and for atypical depression 30.5. Total cholesterol levels strongly correlated with BMI expect for morbidly obese subjects. Cholesterol levels in male depressed subjects were higher than female p=0.02. Increases in total cholesterol did not correlate with severity of depression.

In this population of depressed patients cholesterol levels are increased, not decreased as previously reported. Patients with MDD are overweight and ADD patients are obese. Male cholesterol levels are higher than female. Atypically depressed patients have greater increases than classically depressed patients. Older depressed subjects are significantly increased. Greater severity of depression dies not correlate with increased cholesterol levels. Clinicians should be aware and aggressively treat cholesterol and lipid levels in this population.

### Example 3

### The Effect of SSRIs on Total Cholesterol Levels in Subjects with Classic or Atypical Depression

Major depression has been associated with early death due to cardiovascular mechanisms secondary to increased lipids and/or the metabolic syndrome. Psychiatric drugs have been implicated in deleterious effects on weight, lipids, and glucose. The 5HT₂-D₂ antagonists are especially damaging in this regard with a significant proportion of subjects gaining weight, with complications of high lipid levels and diabetes. Current treatment of Major Depression involves the Specific Serotonin Reuptake Inhibitors (SSRIs) which affect serotonin receptors. Fluoxetine and Sertraline have been shown to have less antidepressant effect in subjects with elevated cholesterol. Further they have been shown to have a cholesterol elevating effect in depressed subjects. The elevation is similar in HDL and LDL cholesterol fractions. The effect in subclasses of depression has not been measured.

Classic depression (MDD) is associated with loss of appetite and insomnia, whereas atypical depression (ADD) is associates with hyperphagia and hypersomnia.

Objective:

To evaluate the pre-treatment cholesterol levels in MDD and ADD populations and to determine the effect of SSRIs on Total cholesterol levels in subjects with MDD and ADD.

Methods:

In 2 eight week clinical studies of the antidepressant effects of SSRIs (fluoxetine and paroxetine) in ADD comprised of 845 subjects and 2 studies of MDD comprised of 602 subjects, safety measurements including non-fasting measurements of cholesterol were observed at baseline and at 8 weeks (endpoint). A JMP-7 one way ANOVA of the data provided results for individual studies which were then analyzed by meta-analysis.

Results:

BMI for MDD was 29.5 and for ADD 30.5 (p<0.001). Untreated ADD (203.1 mg/dL) and MDD subjects (199.1 mg/dL) do not have elevated cholesterol levels compared to NHANES (200.3 mg/dL). At baseline cholesterol levels were elevated in ADD subjects compared to MDD subjects: difference +3.7 mg/dL, p=0.021. At endpoint cholesterol levels in ADD and MDD subjects treated with SSRI are not different from baseline. However, placebo levels decrease resulting in an apparent 5-6 mg/dL difference for both the MDD and ADD groups.

The BMI is statistically higher in ADD than MDD subjects. Untreated ADD subjects have statistically greater cholesterol levels than MDD subjects. Treatment with SSRIs does not elevate cholesterol in either ADD and MDD subjects. Apparent elevations occur in both treatment groups because placebo levels decrease.

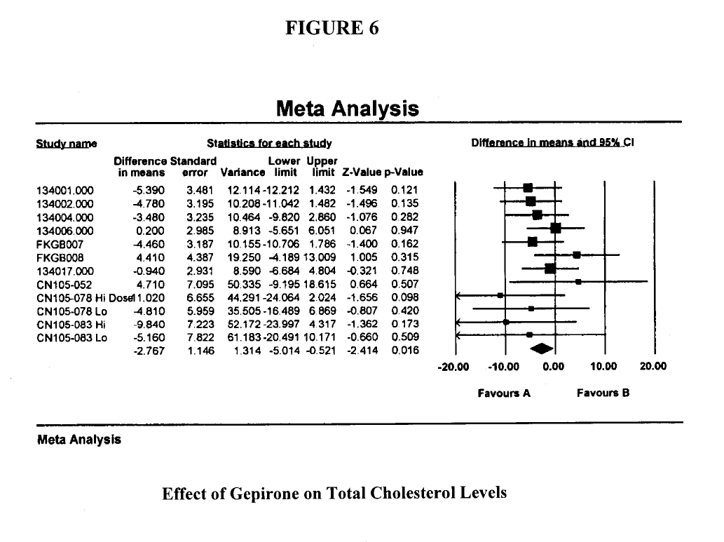

The inventors then decided to examiner the effect of gepirone on total cholesterol levels with the expectation that gepirone would either increase cholesterol levels or have no effect. To their surprise, it was found that gepirone decreased cholesterol levels. The results show that in 10 studies, gepirone has lower total cholesterol levels in 7 of the 10. The two studies that have two gepirone dose groups show both groups with a cholesterol lowering effect. The meta-analysis shows an average drop of 2.8 mg/dL, statistically significant at p=0.016 (See FIG. 6). in the baseline data above it is noted that all the cholesterol levels are within normal limits. (These patients were not selected to have elevated cholesterol levels.)

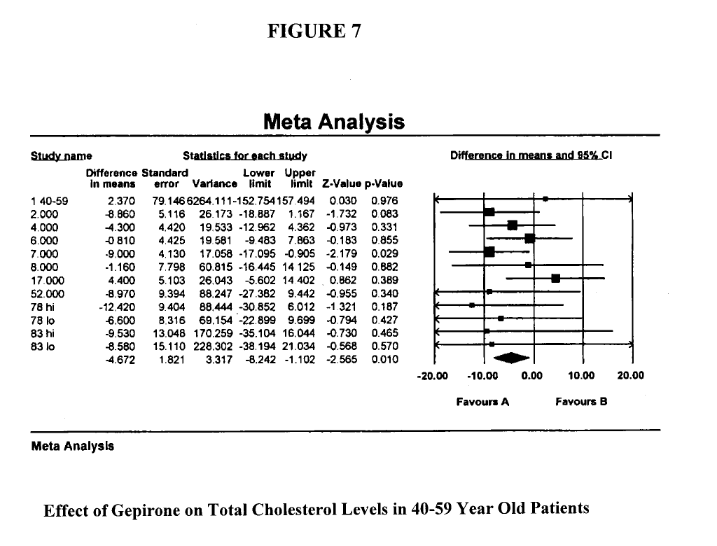

However, the cholesterol levels in depressed populations increase with age. In the 40-59 year range, the average increase was +5.1 mg/dL. So, analysis of the effect of gepirone on cholesterol levels in the 40-59 year range was conducted. The results show that gepirone lowered cholesterol levels in this group by −5 mg/dL, exactly the increase above normal (See FIG. 7). This shows that the effect is clinically significant.

It is noted that the plasma levels of 3-OH gepirone in animals and humans treated with gepirone are greater than that of gepirone. 3-OH gepirone has the same receptor binding, therefore, it is theorized that 3-OH-gepirone accounts for at least some of the effect noted with gepirone.

### Example 4

### Effect of Adatanserin on Cholesterol Levels

Adatanserin is another drug with similar properties. The studies done in adatanserin were largely done on anxiety, not depression. It was not known if the cholesterol levels in subjects with anxiety are elevated. (In the small sample studied below, the levels are not elevated). One paper in subjects with panic disorder claims that paroxetine treatment elevated plasma cholesterol (Kim E J, YU B-H, Increased Cholesterol Levels After Paroxetine Treatment in Patients with panic Disorder. J Clinical Psychopharmacol 2005; 25:597-599). However, to see if adatanserin might also have cholesterol lowering properties, the inventors of the present invention examined a random subset of 103 subjects from study 790-210.

The results show that adatanserin also has cholesterol lowering properties. The results are as follows:

Adatanserin n=76
Total Cholesterol: 204.46 (46.4) mg/dL at baseline
197.46 (44.1) mg/dL after treatment
Change −7.00 mg/dL
   Placebo n=27
   Total Cholesterol 201.54 (36.8) mg/dL at baseline
210.63 (43.1) mg/dL after treatment
Change 8.22 mg/dL
   Difference between adatanserin and placebo: 15.22 mg/dL, p=0.011

Since 3-OH adatanserin is an active metabolite of adatanserin, it is theorized to also have cholesterol-lowering properties.

### Example 5

### Effect of Cholesterol Levels of Efficacy of 5HT_{1A} Receptor Agonists

The effects of cholesterol on gepirone-ER efficacy was investigated. As Gepirone-ER also does not have efficacy in two thirds of the population tested. As such, it was expected that the results for gepirone to be similar to that of Fava. Specifically, the hypothesis that gepirone-ER does not work in subjects with high cholesterol levels was tested by two measures: (1) the number of respnders (50% decrease in HAMD-17) and (2) the HAMD-17 change from baseline score. Corresponding placebo values were generated as well.

The total population used was 10 times greater than the Fava Study addressed in the background section (Sonawalla S B, Papakostas G I, Petersen T J, Yeung A S, Smith M M, Sickinger A H, Gordon J, Israel J A, Tedlow J R, Lamon-Fava S, Fava M, Elevated cholesterol levels associated with nonresponse to fluoxetine treatment in major depressive disorder. Psychosomatics. 2002; 43:310-6). Using the cutoff value of 200 mg/dL, there were 1264 subjects above 200 mg/dL and 1225 subjects below. The average ages of the two FK groups were comparable to those from the Fava data set, with an average age of 42.1 years for subjects over 200 mg/dL, and 34.8 years under 200 mg/dL. The respective average cholesterol levels were 234.3±29.7 and 169.9±21.2, similar to the Fava data.

The results there compared by t-test of odds ratio programs. The results for gepirone were examiner in 8 MDD studies. The low cholesterol group treated with gepirone-ER had 41.2% responders compared to the high cholesterol treated group 43.3% responders, a 1.9% difference favoring the high cholesterol group (p=0.58)(Table 2). When the gepirone ER and placebo groups (Table 2) are compared, the high cholesterol gepirone treated group is 13.6% better than placebo (p=0.002), whereas the difference between the low cholesterol gepirone and placebo group is 0.5%, (p=0.602). However, the over 200 placebo group is 11% less than the under 200 placebo group (p=0.004).

TABLE 2
FK: Gepirone vs. Placebo Treatment, Double-blind, MDD

| | | | HAMD responders | | |
|---|---|---|---|---|---|
| | | | | p-value | |
| | Cholesterol | | | gepirone vs. | p-value |
| Treatment | mg/dL | n | percent | placebo | over vs. under |
| Gepirone | Over 200 | 344 | 43.3% | 0.002 | 0.58 |
| Gepirone | Under 200 | 354 | 41.2% | 0.602 | |
| Placebo | Over 200 | 316 | 29.7% | | 0.004 |
| Placebo | Under 200 | 337 | 40.7% | | |

Using HAMD change from baseline and comparing versus placebo (Table 3), the standard difference in means for the low cholesterol group is −0.02 points favoring gepirone (p=0.656). the high cholesterol gepirone treated group difference in means is −1.77 points favoring gepirone (p=0.001).

TABLE 3
FK: Gepirone vs. Placebo Treatment, Double-blind, MDD

| | Choles- | | HAMD change from baseline | | | |
|---|---|---|---|---|---|---|
| | terol | | gepirone vs. placebo | | over vs. under | |
| Treatment | mg/dL | n | Δ means ± SD | p-value | Δ(Δ means) | p-value |
| Gepirone | Over | 344 | −9.58 ± 7.2 | 0.001 | 0.10 | 0.856 |
| | 200 | | | | | |
| Gepirone | Under | 354 | −9.48 ± 7.4 | 0.656 | | |
| | 200 | | | | | |
| Placebo | Over | 316 | −7.79 ± 7.0 | | 1.67 | 0.003 |
| | 200 | | | | | |
| Placebo | Under | 337 | −9.46 ± 7.4 | | | |
| | 200 | | | | | |

These results suggest both by responder rate p=0.002 and by change from baseline total HAMD-17 p=0.001 that gepirone works better in the high cholesterol group. Gepirone-ER is shown to have a greater effect in the over 200 treated group then the under 200 treated group.

There are differences in the over 200 placebo and under 200 placebo antidepressant effects. This is statistically significant from both HAMD responders and HAMD change from baseline. The reason for this is unknown, however it is noted that the high cholesterol level group is older subjects. Based on the above study and the literature, Gepirone-ER is the only treatment with a greater effect in the over 200 treated group than the under 200 treated group. This improved efficacy is statistically significant.

A surprising result from this data is that gepirone-ER has better antidepressant efficacy in the high cholesterol group than the low cholesterol group.

1. A method of treating an elevated cholesterol level comprising administering a therapeutically effective amount of at least one 5HT_{1A} receptor agonist to a subject in need thereof.

2. The method of claim 1, wherein the 5HT_{1A} receptor agonist is at least one selected from the group consisting of gepirone, ipsapirone, tandospirone, adatanserin, flesinoxan or buspirone.

3. The method of claim 1, wherein the subject in need thereof also suffers from and/or is diagnosed with at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's.

4. The method of claim 1, wherein the 5HT_{1A} receptor agonist is gepirone.

5. The method of claim 1, wherein the 5HT_{1A} receptor agonist is adatanserin.

6. The method of claim 1, wherein the elevated cholesterol level is a cholesterol level that exceeds 200 mg/dL.

7. The method of claim 1, wherein said subject is human.

8. The method of claim 1, wherein said therapeutically effective amount ranges from 20-80 mg per day per day.

9. A method of maintaining a cholesterol level below 200 mg/dL in a subject in need thereof, said method comprising administering a therapeutic effective amount of at least one 5HT_{1A} receptor agonist to said subject.

10. The method of claim 9, wherein the 5HT_{1A} receptor agonist is at least one selected from the group consisting of gepirone, ipsapirone, tandospirone, adatanserin, flesinoxan and buspirone.

11. The method of claim 9, wherein the subject in need thereof suffers from and/or is diagnosed with at least one of the following disorders selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's.

12. The method of claim 9, wherein the 5HT_{1A} receptor agonist is gepirone.

13. The method of claim 9, wherein the 5HT_{1A} receptor agonist is adatanserin.

14. The method of claim 9, wherein said subject is human.

15. The method of claim 9, wherein said therapeutically effective amount ranges from 20-80 mg per day.

16. A method of treating at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's, said method comprising administering to a subject in need thereof an effective amount of at least one 5HT1A agonist, wherein the subject has an elevated cholesterol level.

17. The method of claim 16, wherein the 5HT_{1A} receptor agonist is at least one selected from the group consisting of gepirone, ipsapirone, tandospirone, adatanserin, flesinoxan and buspirone.

18. The method of claim 16, wherein an elevated cholesterol level is a cholesterol level that exceeds 200 mg/dL.

19. A method of treating at least one disorder selected from the group consisting of attention deficit disorder, depression, sexual dysfunction, psychoses, aggressive behavior, alcohol dependence, drug abuse/addiction, obesity, anxiety, sleep disorder, and behavioral/cognitive symptoms of Alzheimer's, said method comprising:
identifying a subject suffering from at least one of the above disorders that has an elevated cholesterol level,
administering to a subject in need thereof an effective amount of at least one 5HT_{1A} agonist.

20. The method of claim 19, wherein the 5HT_{1A} receptor agonist is at least one selected from the group consisting of gepirone, ipsapirone, tandospirone, adatanserin, flesinoxan and buspirone.

21. The method of claim 19, wherein an elevated cholesterol level is a cholesterol level that exceeds 200 mg/dL.
